(19) United States
(12) Patent Application Publication
(10) Pub. No.: US 2016/0175243 A1
(43) Pub. Date: Jun. 23, 2016

(54) NANOROD MATERIALS AND METHODS OF MAKING AND USING SAME

(71) Applicant: UNIVERSITY OF FLORIDA RESEARCH FOUNDATION, INC., Gainesville, FL (US)

(72) Inventors: TANMAY P. LELE, GAINESVILLE, FL (US); FAN REN, GAINESVILLE, FL (US); BENJAMIN G. KESELOWSKY, GAINESVILLE, FL (US); JIYEON LEE, GAINESVILLE, FL (US); ANAND GUPTE, GAINESVILLE, FL (US); BYUNG-HWAN CHU, GAINESVILLE, FL (US); KARL R. ZAWOY, HIGH SPRINGS, FL (US)

(21) Appl. No.: 14/591,729

(22) Filed: Jan. 7, 2015

Related U.S. Application Data
(62) Division of application No. 12/936,491, filed on Dec. 13, 2010, now abandoned.
(60) Provisional application No. 61/049,262, filed on Apr. 30, 2008.

(51) Int. Cl. A61K 9/00; A61K 38/02; A61K 31/366; A61K 33/24; A61K 38/48; C01G 9/02; A61K 31/407

(52) U.S. Cl. CPC A61K 9/00; C01G 9/02; A61K 38/02; A61K 31/407; A61K 33/24; A61K 38/4893; A61K 31/366; Y10S 977/762; Y10S 977/915; B82Y 5/00

(57) ABSTRACT

The subject invention concerns nanorods, compositions and substrates comprising nanorods, and methods of making and using nanorods and nanorod compositions and substrates. In one embodiment, the nanorod is composed of Zinc oxide (ZnO). In a further embodiment, a nanorod of the invention further comprises SiO₂ or TiO₂. In a specific embodiment, a nanorod of the invention is composed of ZnO coated with SiO₂. Nanorods of the present invention are useful as an adhesion-resistant biomaterial capable of reducing viability in anchorage-dependent cells.

### CROSS-REFERENCE TO RELATED APPLICATIONS

The present application is a divisional of U.S. application Ser. No. 12/936,491, filed Dec. 13, 2010, which is the National Stage of International Application Number PCT/US2009/002686, filed Apr. 30, 2009, which claims the benefit of U.S. Provisional Application Ser. No. 61/049,262, filed Apr. 30, 2008, each of which is hereby incorporated by reference herein in its entirety, including any figures, tables, nucleic acid sequences, amino acid sequences, and drawings.

### GOVERNMENT SUPPORT

This invention was made with government support under grant number N000140710982 awarded by the Office of Naval Research and under grant number DAAD19-01-1-0603 awarded by the Army Research Office. The government has certain rights in the invention.

### BACKGROUND OF THE INVENTION

The success of implanted devices such as orthopedic implants, cardiovascular prosthesis and neural electrodes is affected by the ability of cells to interact with the exposed device material. Because properties such as surface topology are stable features of the surface, compared to chemical modifications which may be degraded over time, there has been immense interest in directing cell behavior by controlling the topology of materials (Andersson et al.; Chen et al.; Choi et al.; Dalby et al. (2002); Dalby et al. (2005); Diehl et al.; Flemming et al.; Gonsalves et al.; Karuri et al.; Lenhert et al.; Liliensiek et al.; Ma et al.; Re et al.; Teixeira et al. (2003); Teixeira et al. (2006); Yim et al.). Cells have been found to respond differently to smooth surfaces compared to materials with micro or nano-scale roughness in a cell type dependent manner (Yim et al.; Khang et al.; Spatz et al.; Washburn et al.).

Cells adhere to and spread on materials by assembling specialized supramolecular protein complexes called focal adhesions (Bershadsky et al.). Focal adhesion assembly occurs through the ligation of integrin receptors to immobilized ligands such as fibronectin and subsequent clustering of ligated receptors. Variations in nano-scale topography of the substrate can modulate nano-scale integrin ligation and clustering, resulting in changes in adhesion assembly (Spatz et al.; Arnold et al.; Cavalcanti-Adam et al.; Girard et al.). Because focal adhesions are signaling complexes (Bershadsky et al.), such changes in adhesion assembly can alter signaling pathways resulting in the regulation of cell behavior. Indeed, cell behaviors such as motility and adhesion, proliferation and differentiation have been found to be exquisitely sensitive to nano-scale topography of the substrate. A study showed that mesenchymal cells on Si nanowires survived for days, and even differentiated despite being impaled on Si nanowires (Kim et al. 2007a; Pearton et al.).

Stents are often used for palliation of inoperable esophageal, gastric, hepatobiliary, colon, pancreatic and pulmonary cancers. A key challenge in the use of stents is tumor cell in-growth in the stent, which causes re-obstruction (occlusion), requiring early endoscopic removal or even surgery. Untreated, this stent occlusion can lead to difficulty in swallowing (dysphagia), difficulty breathing (dsypnea), intestinal obstruction, jaundice with severe infection and possible death. This introduces severe complications in the management of the cancers, and greatly reduces the quality of the patient's life. One recent approach is to cover the metallic stent with plastic, which attempts to prevent tumor cell adhesion. But this causes undesirable migration of the stent owing to the smooth stent surface. Thus, to improve stent performance, new methods are needed that will minimize tumor cell adhesion, survival and proliferation on the stent.

### BRIEF SUMMARY OF THE INVENTION

The subject invention concerns nanorods (also referred to as nanoposts and nanocolumns), compositions and substrates comprising nanorods, and methods of making and using nanorods and nanorod compositions and substrates. In one embodiment, a nanorod of the invention is composed of zinc oxide (ZnO). In another embodiment, a nanorod of the invention is composed of titanium dioxide (TiO₂), silicon (Si), indium nitride (InN), or gallium nitride (GaN). In a further embodiment, a nanorod of the invention further comprises a coating of SiO₂ and/or TiO₂. In a specific embodiment, a nanorod of the invention is composed of ZnO coated with SiO₂ and/or TiO₂. Cell adhesion and survival was reduced on surfaces having a monolayer of upright ZnO nanorods of the present invention. Owing to the uniform distribution of the nanorod monolayer, cells were not able to attach to any flat portion of the substrate. Initial adhesion, lamellipodia formation, dynamic cell spreading and cell survival at 24 hours of three different cell types was greatly reduced on nanorod covered substrates. These results show that upright nanorod type structures can be used for minimizing cell adhesion and survival.

The subject invention also concerns methods for preparing a substrate surface comprising nanorods of the present invention. In one embodiment, a substrate surface is prepared with nanorods composed of ZnO. In another embodiment, a substrate surface is prepared with nanorods composed of TiO₂, Si, InN, or GaN. In a further embodiment, a substrate surface is prepared with a nanorod that comprises a coating of SiO₂ and/or TiO₂. In a specific embodiment, the substrate surface is prepared with a nanorod composed of ZnO coated with SiO₂ and/or TiO₂. The substrate surface is typically prepared such that the nanorods attached to the surface are generally uniformly spaced apart, densely packed, and/or vertically aligned in a monolayer.

The subject invention also concerns substrates having surfaces with nanorods of the present invention attached thereto. In one embodiment, the substrate is prepared such that the nanorods attached to the surface are generally uniformly spaced apart, densely packed, and/or vertically aligned in a monolayer. In one embodiment, the substrate surface comprises a nanorod composed of ZnO. In another embodiment, the substrate surface comprises a nanorod composed of TiO₂, Si, InN, or GaN. In a further embodiment, a substrate surface comprises a nanorod of the invention with a coating of SiO₂ and/or TiO₂. In a specific embodiment, the substrate surface comprises a nanorod composed of ZnO coated with SiO₂ and/or TiO₂.

The subject invention also concerns any device, product, apparatus, structure, or material that is capable of or is to be implanted in a living body, such as a human body, wherein the device, product, apparatus, structure, or material is coated on all or a portion of the device, product or apparatus, structure, or material with nanorods of the present invention.

The subject invention also concerns any device, product, apparatus, structure, or material on which cells or an organism might otherwise attach and grow, wherein the device, product, apparatus, structure, or material is coated on all or a portion thereof with nanorods of the present invention to inhibit and/or reduce cellular adhesion, growth, and/or survival to the surface of a substrate of the device, product, apparatus, structure, or material.

The subject invention also concerns methods for delivery of chemicals and drugs into cells. In one embodiment, nanorods of the invention are coated with or attached to a chemical or drug compound and contacted with a cell.

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

FIGS. 1A, 1B, 1C-1, and 1C-2 show the morphology of ZnO nanorods and flat substrate. FIGS. 1A-1B are SEM images of ZnO nanorods indicating a uniform monolayer of ZnO (FIG. 1A; scale bar is 2 μm, and the upright growth of nanorods (FIG. 1B; scale bar is 500 nm). The diameter of nanorods was ˜50 nm and the height was ˜500 nm. FIGS. 1C-1 and 1C-2 are AFM images of ZnO flat substrate. The surface roughness was approximately 1.33 nm indicating that this substrate is much smoother than the nanorods and can be used for comparisons of cell behavior between nanorods and smooth surfaces.

FIG. 2 shows that cells do not assemble stress fibers or focal adhesions on nanorods. Fluorescent micrographs of NIH 3T3, HUVEC, and BCE cells stained for vinculin (green) and F-actin (red) on glass, ZnO flat substrate and ZnO nanorods. The cell spreading area is greatly reduced, and focal adhesions and stress fibers are not visible in cells cultured on the nanorods. Scale bar is 20 μm.

FIGS. 3A-3D show that total cell number and number of live adherent cells are reduced on nanorods. The average number of cells adherent on each substrate, the number of adherent live cells (stained with calcein AM) and adherent dead cells (stained with EthD-1) were quantified in three cell types (FIGS. 3A-3C) by pooling data from five different images per cell type and condition. Bars indicate standard error of the mean. * indicates statistically significant differences with p<0.01 between the number of cells on ZnO nanorods and flat substrates (n>50 for HUVEC, n>30 for BCE, n>300 for fibroblasts). The number of live cells on ZnO nanorods normalized by the number of live cells on ZnO flat substrates (FIG. 3D). The results show that the decrease of the number of live adherent cells on the nanorods is robust across three different cell types, with a large effect demonstrated in endothelial cells (HUVEC, BCE) than fibroblasts (NIH 3T3).

FIGS. 4A-4D show that cells cannot assemble lamellipodia on nanorods. Representative SEM images of NIH 3T3 fibroblasts on ZnO nanorods. Most of cells on ZnO nanorods were round and they did not form lamellipodia (FIGS. 4A and 4B). Scale bars in FIGS. 4A and 4B are 3 μm and 1 μm, respectively. Filopodia-like structures were observed in some cells on nanorods (white arrows in inset) along with thin processes (black arrows) (FIGS. 4C and 4D). Scale bars in FIGS. 4C and 4D are 5 μm and 2 μm, respectively.

FIGS. 5A and 5B show dynamic cell spreading is altered on nanorods. Phase contrast imaging of HUVECs spreading on glass and ZnO nanorods. In FIG. 5A, cell spreading HUVECs is accompanied by lamellipodia formation (white arrows) and is complete in approximately five hours. In FIG. 5B, cells on nanorods do not spread, and do not develop any lamellipodia. Scale bar is 20 μm.

FIGS. 6A-6C. In FIG. 6A, purity of the macrophage culture was determined to be ˜90% as determined by immunofluorescent quantification of F4/80 and CD11b, murine macrophage markers by flow cytometric analysis. FIGS. 6B and 6C are SEM images of ZnO nanorods and flat substrate. Scale bar is 200 μm.

FIG. 7 shows time lapse image of macrophage seeded on fibronectin coated ZnO nanorods. Phase contrast imaging of macrophages on ZnO nanorod surface over 13 hours post seeding. As compared to glass (data not shown) the macrophages start rounding up instead of spreading. Scale bar is 20 um.

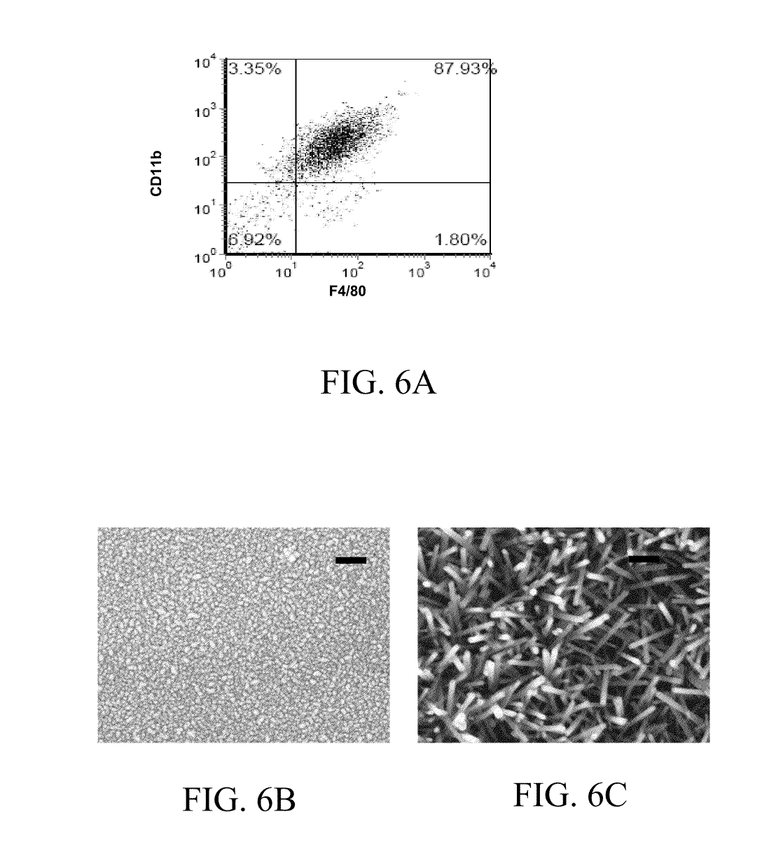

FIG. 8 shows total numbers of cells adherent and viable on ZnO flat substrate and nanorods is reduced as compared to glass at 3 hours post seeding. The average number of live (stained with calcein AM) and dead cells (stained with 7-AAD) on the 3 substrates—glass, ZnO flat substrate and ZnO nanorods was quantified by pooling data from 6 samples from 3 separate runs. Three images were taken per sample. Bars indicate standard error of means. Tukey's Honestly-Significant-Difference Test was performed to evaluate statistical significance between ZnO nanorod and the control glass and ZnO flat substrate. * Indicates statistically significant difference (p<0.05) from all other conditions. Embedded symbols represent significance among live/dead groups, while symbols above cumulative bars represent significance among total adherent cell numbers.

FIG. 9 shows total numbers of cells adherent and viable on ZnO flat substrate and nanorods is reduced as compared to glass at 16 hours post seeding. The average number of live (stained with calcein AM) and dead cells (stained with 7-AAD) on the 3 substrates—glass, ZnO flat substrate and ZnO nanorods was quantified by pooling data from 7-8 samples from 4 separate runs. Three images were taken per sample. Bars indicate standard error of means. Tukey's Honestly-Significant-Difference Test was performed to evaluate statistical significance between ZnO nanorod and the control glass and ZnO flat substrate. * Indicates statistically significant difference (p<0.05) from all other conditions. Embedded symbols represent significance among live/dead groups, while symbols above cumulative bars represent significance among total adherent cell numbers.

FIGS. 10A-10B. FIG. 10A shows the setup to determine cytotoxicity of ZnO when cells are not present in contact with it. Cells are cultured at the bottom of 6 well plate. The substrates to be tested are inverted in the well as shown with the help of Viton O-rings so that they are bathing in the same media as the cells. In FIG. 10B, total number of live cells adherent on 6 well plates is greatly reduced when ZnO flat substrates and ZnO nanorods are inverted in the media as compared to inverted glass. The average number of live adherent cells on the 3 substrates—glass, ZnO flat substrate and ZnO nanorods was quantified by pooling data from 8 samples from 2 separate runs. Three images were taken per sample. Bars indicate standard error of means. Tukey's Honestly-Significant-Difference Test was performed to evaluate statistical significance between ZnO nanorod and the control glass and ZnO flat substrate. * Indicates statistically significant difference (p<0.05) from the number of cells on glass

FIGS. 11A-11C: Zinc oxide coating on implanted discs prevents formation of acellular fibrous capsule around disc indicating unresolved inflammation. Verhoeff-van-Geisson stained sections (collagen pink;cell nuclei:dark blue) of tissue response to implanted biomaterials. FIG. 11A: PET disc not coated with ZnO nanorods has a smooth capsule formation around discs indicated by the arrow. FIG. 11B: PET disc coated with ZnO nanorods does not have a smooth capsule formation around discs. Instead it has an accumulation of cells (indicated by arrow) which may primarily macrophages which were recruited to the site of implantation. FIG. 11C: SEM image of nanorods coated on the PET discs.

FIGS. 12A-12C show the morphology of nanorods. FIG. 12A shows a TEM image of SiO₂ deposited ZnO nanorods. Black arrows indicate SiO₂ thin film with a 50 Å thickness. ZnO nanorods are encapsulated by SiO₂. FIG. 12B is a scanning electron microscopy (SEM) image of nanorods on glass. White arrows indicate the spacing between nanorods. The spacing between nanorods ranges from 80 to 100 nm. FIG. 12C is a SEM image of a monolayer of nanorods. Upright nanorods were covered on the underlying glass substrate uniformly.

FIGS. 13A-13D show fluorescent microscopic images of HUVEC and NIH 3T3 on glass and nanorods. In FIGS. 13A and 13C, HUVEC and NIH 3T3 on glass assemble focal adhesions stained with vinculin (green) and actin stress fibers (red). Nuclei were stained with DAPI (blue). In FIGS. 13B and 13D, HUVEC and NIH 3T3 on nanorods are unable to spread and assemble focal adhesions and stress fibers.

FIGS. 14A and 14B show the average area of cell spreading on glass and nanorods. FIG. 14A shows HUVEC on glass and nanorods (n>170). FIG. 14B shows NIH 3T3 on glass and nanorods (n>110). * indicates p<0.005. Bar indicates standard error of the mean (SEM). The data were pooled from three independent experiments.

FIGS. 15A and 15B show SEM images of patterned nanorods. FIG. 15A is an optical microscope image with 400 X objective. FIG. 16B is a SEM image.

FIGS. 16A-16E show NIH 3T3 fibroblasts on patterned nanorods. FIGS. 16A and 16B show phase contrast and fluorescent microscopic images showing that NIH 3T3 fibroblasts preferably attached on glass. Cells are stained for actin (red), vinculin (green) and nucleus (blue). FIGS. 16C-16E show differential interference contrast and fluorescent microscope images. Cells were confined on the flat circular regions. Dashed lines indicate the edge of patterns.

FIGS. 17A-17C show time-lapse phase microscope images of NIH 3T3 on patterned nanorods. Black arrows indicate the direction of cell motility. Cells are observed to move from glass to glass by spanning intervening nanorods (FIG. 17A), or continuously explore the nanorod environment at the edges of the circle (FIG. 17B and FIG. 17C). Arrows indicate direction of motion.

FIGS. 18A and 18B show cell attachment and viability on nanorods. FIG. 18A shows the ratio of the number of attached cells on nanorods to that on glass. FIG. 18B shows the ratio of the number of live cells on nanorods to that on glass. (n>2500 for HUVECs, n>1500 for NIH 3T3. Bar indicates SEM. Cells are considerably reduced in numbers on nanorods.

### DETAILED DESCRIPTION OF THE INVENTION

The subject invention concerns nanorods, compositions and substrates comprising nanorods, and methods of making and using nanorods and nanorod compositions and substrates. In one embodiment, a nanorod of the invention is composed of zinc oxide (ZnO). In another embodiment, a nanorod of the invention is composed of TiO₂, Si, InN, or GaN. In a further embodiment, a nanorod of the invention further comprises SiO₂ and/or TiO₂. In a specific embodiment, a nanorod of the invention is composed of ZnO coated with SiO₂ and/or TiO₂. In a more specific embodiment, a nanorod of the invention is composed of ZnO coated with SiO₂. Advantageously, nanorods of the invention can be prepared or provided on a substrate or surface at room temperature, thereby allowing for application of nanorods to polymers and other materials that could be destroyed by excessive heat. Nanorods of the invention can be applied to surfaces using industrial dip coating processes and, thus, can be applied to surfaces and materials having complicated geometries.

One aspect of the present invention concerns methods for preventing, inhibiting and/or reducing cellular adhesion, growth, and/or survival to a substrate surface. The present invention can help prevent or control fibrosis around an implanted biomaterial that comprises nanorods of the invention. In one embodiment, a substrate surface is provided with a coating of nanorods of the present invention. In an exemplified embodiment, the nanorods are composed of ZnO. In another embodiment, a nanorod of the invention is composed of TiO₂, Si, InN, or GaN. In a further embodiment, a nanorod of the invention further comprises a coating of SiO₂ and/or TiO₂. In a specific embodiment, the nanorods are composed of ZnO coated with SiO₂ and/or TiO₂. The nanorods are advantageously provided in densely packed, vertically aligned monolayers. The substrate can be any substrate for which the inhibition or reduction of cellular adhesion, survival, and/or growth is desired. Substrates contemplated within the scope of the invention that can be coated with nanorods of the invention include, but are not limited to, glass, plastics (such as polyethylene and polypropylene), fiberglass, wood, rubber, metals and alloys, ceramics, cloth, polymers, concrete, silicon, and paint. Cells contemplated within the scope of the invention that can be inhibited using nanorods of the invention include, but are not limited to, cells from animals such as mammals (including humans); plants; algae; fungi such as mold; and bacteria. In one embodiment, the cell is a macrophage. In another embodiment, the cell is a fibroblast or an endothelial cell. In a further embodiment, the cell is an osteoclast or osteoblast. In a still further embodiment, the cell is a tumor or cancer cell. The tumor or cancer cell contemplated within the scope of the invention include, but is not limited to, cells from tumors or cancers of the bone, breast, kidney, mouth, larynx, esophagus, stomach, testis, cervix, head, neck, colon, ovary, lung, bladder, skin, liver, muscle, pancreas, prostate, blood cells (including lymphocytes), and brain.

In one embodiment, nanorods of the invention are coated or conjugated with one or more cellular toxins. In a specific embodiment, the cellular toxin is only released from the nanorod in an intracellular environment, e.g., when a cell engulfs or is impaled on a nanorod. Examples of toxins include, but are not limited to, ricin, mitomycin C, cisplatin, botulinum toxin, anthrax toxin, aflatoxin, and the like. In one embodiment, the toxin is attached to a cleavable moiety that can be cleaved by molecules (e.g., by an enzyme) present inside the cell.

In a further embodiment, nanorod surfaces could be designed and engineered or functionalized with a moiety to bind to or to act as an attractant for a target cell, compound, molecule, etc. (e.g., probiotics, beneficial bacteria, drugs, etc.). A specific molecule or surface structure of target bacteria would have strong affinity for the nanorods zones while other bacteria would not. A medical device could be pretreated in specific zones (Pretreated Zones) with probiotics or patient favorable bacteria or with drugs that could be applied and thereby defend against other infections. In another embodiment, a nanorod surface could be designed to enhance surface adhesion of inorganic and organic materials, drugs, drug delivery mechanisms in oral, vascular or similar applications such as by the use of heparin coatings, or coatings that typically elude in a short period of time. In one embodiment, a nanorod of the invention is functionalized with an antibody or an antigen binding fragment thereof, receptor, peptide, nucleic acid, or aptamer that has binding specificity for a target molecule (e.g., an antigen on a target cell or bacterium).

The subject invention also concerns methods for preparing a substrate surface comprising nanorods of the present invention. In one embodiment, a substrate surface is prepared with nanorods composed of ZnO. In another embodiment, a substrate surface is prepared with nanorods composed of TiO₂, Si, InN, or GaN. In a further embodiment, a substrate surface is prepared with a nanorod that comprises a coating of SiO₂ and/or TiO₂. In a specific embodiment, the substrate surface is prepared with a nanorod composed of ZnO coated with SiO₂ and/or TiO₂. The substrate surface is typically prepared such that the nanorods attached to the surface are generally uniformly spaced apart, densely packed, and/or vertically aligned in a monolayer. However, the height, spacing, and distribution of nanorods on a substrate surface can be modulated as described herein. Nanorods of the invention can be applied to substrate surfaces in zones or regions. These zones or regions are resistant to cellular growth and/or biological contamination. The subject invention also concerns substrates having surfaces with nanorods of the present invention attached thereto. In one embodiment, the substrate is prepared such that the nanorods attached to the surface are generally uniformly spaced apart, densely packed, and/or vertically aligned in a monolayer. In one embodiment, the substrate surface comprises a nanorod composed of ZnO. In another embodiment, the substrate surface comprises a nanorod composed of TiO₂, Si, InN, or GaN. In a further embodiment, a substrate surface comprises a nanorod of the invention with a coating of SiO₂ and/or TiO₂. In a specific embodiment, the substrate surface comprises a nanorod composed of ZnO coated with SiO₂ and/or TiO₂. Nanorod coatings of the invention typically comprise nanorods that are from about 100 nm to about 5 μm in height, and are from about 5 nm to about 150 nm in diameter. In a specific embodiment, the nanorods are about 500 nm in height and about 50 nm in diameter. In one embodiment, the nanorods can be hexagonal-shaped (viewed in cross-section or needle-shaped. Typically, nanorod coatings of the invention have nanorods spaced apart from between about 5 nm to about 150 nm. In a specific embodiment, the spacing between nanorods is on average about 100 nm. The density of nanorods on the surface of a substrate can be from about 10 rods per square micron to about 1000 or more rods per square micron. In a specific embodiment, the density of nanorods is about 200 to 400 rods per square micron, or about 100 to 200 rods per square micron, or about 110 to about 130 rods per square micron. However, the height, spacing, and distribution of nanorods on a substrate surface can be modulated as described herein. Nanorods of the invention can be applied to substrate surfaces in zones or regions. These zones or regions would be resistant to cellular growth and/or biological contamination.

In a specific embodiment, the substrate contemplated for use in the invention is a substrate for use on an animal body and/or in implantation in an animal body. In one embodiment, the substrate is a biocompatible substrate. In a further embodiment, the substrate is on or part of a material or scaffold for tissue engineering. In another embodiment, the substrate is one that comes into contact with cells or bodily fluids outside of an animal body, for example, in an in vitro application or situation, such as substrates in a heart-lung machine or a kidney dialysis machine. In another embodiment, the substrate is one used for tissue culture wherein inhibition of attachment or adhesion of cells to the substrate is preferred. In a specific embodiment, the substrate is on a tissue culture flask, plate, petri dish, or pipette. In a further embodiment, the substrate is one used on medical products and devices, such as a stent, catheters, bandages, wound dressing and wound treatment materials, stitches, electrical leads, intravascular needles, shunts and drainage tubes, pacemakers, heart valves, aortic prosthesis, ocular implants such as lenses, orthopedic implants such as hip joint or knee implants, implantable drug delivery devices such as insulin or hormone delivery devices, endotracheal tubes, vascular access ports, and the like. Stents contemplated within the scope of the invention include cardiovascular stents and gastrointestinal stents. In a still further embodiment, the substrate is one that is used in food preparation, such as cutting boards and utensils.

The subject invention also concerns any device, product, apparatus, structure, or material that is capable of being or is to be implanted on or in a living body, such as a human body, wherein the device, product, apparatus, structure, or material is coated on all or a portion of the device, product or apparatus, structure, or material with nanorods of the present invention. Examples include medical devices such as bandages, wound dressing and wound treatment materials, catheters, pacemakers, stents, heart valves, aortic prosthesis, ocular implants such as lenses, orthopedic implants such as hip joint or knee implants, implantable drug delivery devices such as insulin or hormone delivery devices, and the like. In one embodiment, the coatings of nanorods of the invention on devices and products can prevent or inhibit cell adhesion and survival, inhibit or prevent fibrotic encapsulation, inhibit or prevent the growth of bacteria, and inhibit or prevent the growth of biofilms. In a specific embodiment, a stent is coated with a nanorod composed of ZnO, optionally coated with SiO₂ and/or TiO₂. Stents contemplated within the scope of the invention include, but are not limited to, those for palliation of tumors and cancers in a patient, and those for cardiovascular and gastrointestinal use. Nanorod coated stents of the invention can prevent or reduce platelet adhesion and/or prevent or minimize fibrotic encapsulation by modulating adhesion of macrophages. In another specific embodiment, a bandage or wound dressing material is coated with a nanorod composed of ZnO, optionally coated with SiO₂ and/or TiO₂.

The subject invention also concerns any device, product, apparatus, structure, or material on which cells or an organism might otherwise attach and grow, wherein the device, product, apparatus, structure, or material is coated on all or a portion thereof with nanorods of the present invention to inhibit and/or reduce cellular adhesion, growth, and/or survival to the surface of a substrate of the device, product, apparatus, structure, or material. For example, in one embodiment, nanorods of the present invention are provided on the walls, floors, or lining of a fresh-water or marine aquarium. In another embodiment, the hull of a watercraft or boat vessel is provided with a coating of nanorods of the invention. In still another embodiment, a dock piling or pier, and/or the hardware associated therewith, is provided with a coating of nanorods of the invention. In another embodiment, a surface that is exposed to damp or wet conditions that favor the growth of organisms (e.g., mold, mildew, and/or fungus), such as a shower, tub, toilet, urinal, or sink is provided with a surface coating of nanorods of the invention. In one embodiment, grout or caulk is coated with the nanorods. In another embodiment, filter elements, such as for a gas (e.g., air) or liquid (e.g., water) filter, are coated with nanorods of the invention. In a specific embodiment, the coating of nanorods is provided on the filter element of a mask such as worn in a hospital or surgical environment. In another embodiment, the substrates of a medical device, or parts or portions thereof, that come into contact within cells or bodily fluids of a subject or patient is provided with a surface coating of nanorods of the invention. Examples of medical devices contemplated include heart-lung machines and kidney dialysis machines.

The subject invention also concerns methods for delivery of chemicals and drugs into cells. In one embodiment, nanorods of the invention are coated with or attached to a chemical or drug compound and contacted with a cell. The nanorods are engulfed by the cells and the chemical or drug is thereby delivered into the cell. In one embodiment, the drug or chemical can be attached or coated to the nanorod via a linker molecule that can be cleaved (e.g., by enzymes present in a cell) once the nanorod is inside the cell, thereby releasing the drug or chemical from the nanorod. In one embodiment, the drug is a synthetic compound such as a pharmaceutical. In another embodiment, the drug or chemical is biologically-derived such as a protein, peptide, or nucleic acid such as DNA or RNA. Thus, one aspect of the subject invention concerns methods for transforming cells with a nucleic acid, comprising contacting the cells with a nanorod(s) of the invention coated with the nucleic acid to be used to transform the cell, wherein the nucleic acid is released from the nanorod once inside the cell.

The subject invention also concerns methods for synthesizing a nanorod of the present invention on the surface of a substrate. In one embodiment, a solution of zinc acetate is mixed with a base solution (such as NaOH) for several hours with heating. In one embodiment, the solutions are mixed for 1 to 3 hours at between 50° C. to 70° C. Zinc oxide nanoparticles that form are then coated onto a substrate, followed by heating the coated substrate. In one embodiment, the ZnO particles are spin coated onto the substrate. The heating can be from 100° C. to 200° C. The coated substrate is then contacted with an aqueous nutrient solution that comprises about 20 mm zinc nitrate hexahydrate (Zn(NO₃)₂.6H₂O) and about 20 mm hexamethylenetriamine (C₆H₁₂N₄) (Kang et al., 2007). The growth of nanorods can be arrested by removing the substrate from the nutrient solution, rinsing (e.g., with water), and drying. Optionally, the nanorods can be coated with SiO₂ or TiO₂. In one embodiment, SiO₂ is deposited on the nanorods using a plasma enhanced chemical vapor deposition system using N₂O and SiH₄ as precursors. Patterns of nanorods of the invention can be produced on a substrate surface using conventional photoresist (PR) lithography.

Nanorods of the invention can be used on substrates to limit biological contamination of products commonly used in non-sterile environments:

a. Resist the growth of bacteria or other contaminants in triage situations.

b. Permit the application and extended use of medical devices in non-sterile environments such as bandages, stitches, catheters, etc.

c. Reduce the frequency of changing or redressing wounds or replacing a medical device. For example, replacing the electrical leads coming out from a patient's body and connecting to an external power supply for a Ventricular Assist Device.

Nanorods of the invention can be designed with conductive or semi-conductive properties. The nanorods can be grown on silicon or other substrates or on top of a circuit pathway. Application of voltage and current to the circuit pathways can energize the nanorods and surrounding surfaces. When alternating or direct current is applied to the circuit, the nanorod surface can exhibit the following:
a. Perform surface sterilization or decontamination of the surface through the redistribution of charged particles over a gas of liquid medium.
b. Perform surface sterilization or decontamination of the surface through generation of surface plasma energy generated by a Dielectric Barrier Discharge or similar technique where the electrode and counter-electrode could be nanorods grown on the surface. Micro- and nano-plasma reactions could be used to perform selective sterilization or cellular disruption on the surface at pre-programmed locations.

The subject invention also concerns smart medical devices utilizing surfaces or zones composed of nanorod electrodes or functionalized nanorod surfaces that can be used to detect environmental conditions, the level of cellular activity, or other events occurring at specific locations on the surface of the device. Information would be passed back to the device or user for interventions such as: change out the device; administer a drug; alert user about possible contamination; or take a predetermined action.

Nanorods of the invention can also be used in military applications. In one embodiment, nanorods of the invention are used to inhibit the adhesion and growth of biological agents and biologically active materials. Nanorods can also be used with other chemical warfare agents to provide a protective effect.

The synthesis of nanorods composed of TiO₂, Si, InN, and GaN has been described in Cai et al. (2007), Jia et al. (2007), Joo et al. (2005), Kryliouk et al. (2007), Kuo et al. (2005), Liliental-Weber et al. (2007), Patzig et al. (2008), Tanemura et al. (2006), and Yan et al. (2008).

As shown herein, the adhesion and viability of fibroblasts, umbilical vein endothelial cells, and capillary endothelial cells are greatly altered on ZnO nanorods of the invention. Cells adhered less and spread less on ZnO nanorods than on the corresponding ZnO flat substrate. Scanning electron microscopy indicated that cells were not able to assemble lamellipodia on nanorods. Time-lapse phase contrast imaging showed that cells initially adherent to nanorods are unable to spread. This suggests that the lack of initial spreading on ZnO nanorods may cause cell death.

The results herein indicate a lack of focal adhesion assembly in cells cultured on ZnO nanorods of the invention. In one embodiment, the spacing between the ZnO nanorods of the invention is approximately 100 nm. Recent work by Arnold et al. showed that focal adhesion assembly requires that the spacing between ligated integrins be less than 70 nm (Arnold et al.). Thus, it is possible that integrin clustering does not occur effectively in cells on ZnO nanorods and therefore prevents focal adhesion assembly. Cells on nanorods of the invention also have no visible lamellipodia. As initial adhesion is required to polymerize actin filaments (Lauffenburger et al.), the lack of lamellipodia is probably due to an inability of cells to establish strong initial adhesion to the substrate, thereby altering the dynamics of cell spreading. The observations described herein of altered cell spreading dynamics are consistent with observations by Cavalcanti-Adam et al. who observed similar behavior on RGD (Arginine-Glycine-Aspartic acid) nanopatterned substrates (Cavalcanti-Adam et al.). Results herein can therefore be explained by a mechanism in which abnormal assembly of focal adhesions due to an inability to cluster integrins contributes to decreased cell spreading on nanorods. Because a lack of cell spreading can cause cell death in each of the cell types studied here (Chen et al.; Re et al.), decreased spreading is associated with decrease in cell survival on nanorods of the invention.

It is interesting to contrast the results herein with the work of Kim et al. (2007a). Kim et al. found that nanowires are engulfed by cells, but do not induce apoptosis. Because the nanowires were sparse in the Kim et al. study (20-30 nanowires exposed to each cell), it is likely that cells attach to the flat portions of the substrate and therefore survived. In the experiments of the present invention, each cell was exposed to 60,000 to 150,000 nanorods. Thus, it is possible that a large number of nanorods were engulfed by cells. If this is the case, then toxicity due to nanorod engulfment may cause cell death. Phagocytosed ZnO nanoparticles have been reported to be cytotoxic in vascular endothelial cells (Gojova et al.). The work by Kim et al. showed that DNA immobilized on Si nanowires could be delivered into cells (Al-Hilli et al.). Thus, the efficiency of ZnO nanorods in preventing cell survival can be further enhanced by chemically conjugating toxins to the surface, and delivering these into the cell through penetration and subsequent cleavage.

The nanorod aspect ratio probably plays an important role in the observed response. For example, Curtis and coworkers do not report a large decrease in cell survival, although they also observed decreased cell spreading on nanoposts (Al-Hilli et al.). The diameter in these studies was 100 nm and the height was 160 nm. Curtis et al. report that nanocolumns are not engulfed by cells. As the aspect ratio of nanorods of the invention is more similar to Kim and co-workers (Kim et al., 2007a) where the nanowires were engulfed by cells, this could be another reason for the decreased cell survival in experiments described herein. Additionally, the observations herein of reduced cell adhesion and survival on nanorods are consistent with at least one recent study which employed an aspect ratio similar to the one used in this paper (Choi et al.).

Controlling cell behavior with biomaterials is necessary for the success of tissue engineering scaffolds, biomedical implants and implanted drug delivery devices. Collectively, the results herein indicate that ZnO nanorods of the invention can be used as an adhesion-resistant biomaterial capable of inducing death in anchorage-dependent cells.

Densely packed upright SiO₂ nanorods were created by coating hydrothermally grown ZnO nanorods with a thin layer of SiO₂ using the plasma enhanced chemical vapor deposition (PECVD) technique. It was found that cell numbers were greatly reduced by nearly two orders of magnitude for fibroblasts and an order of magnitude for HUVECs on SiO₂ coated nanorods. Those cells that were adherent were unable to survive on the nanorods. When cultured on a patterned surface where flat circular areas were surrounded by dense nanorods, cells spent the majority of time confined on the flat areas. This indicates that patterning nanorods is a novel approach to pattern cell adhesion.

### MATERIALS AND METHODS FOR EXAMPLES 1-5

Fabrication of ZnO nanorods. ZnO nanorods were made by a solution-based hydrothermal growth method (Greene et al.; Kang et al.; Pacholski et al.). First, ZnO nanoparticles were prepared by mixing 10 mM zinc acetate dehydrate (Sigma Aldrich, St. Louis, Mo.) with 30 mM of NaOH (Sigma Aldrich, St. Louis, Mo.) at 58° C. for 2 hours. Next, ZnO nanoparticles were spin-coated onto the substrate several times and then post-baked on a hot plate at 150° C. for better adhesion. The substrate with these ‘seeds’ was then suspended upside down in a Pyrex glass dish filled with an aqueous nutrient solution. The growth rate was approximately 1 μm per hour with 100 ml aqueous solution. To arrest the nanorod growth, the substrates were removed from solution, rinsed with de-ionized water and dried in air at room temperature.

Preparation of substrates for cell culture. For control substrate, we used 22 mm square glass cover slips (Corning, Inc., Lowell, Mass.) and ZnO flat substrates (Cermet, Inc., Atlanta, Ga.). Before use, each substrate was sterilized with UV for 5 minutes and cleaned in 70% ethanol and de-ionized water. After drying substrates in air at room temperature, they were treated with 5 μg/ml human fibronectin (FN) (BD biosciences, Bedford, Mass.). After overnight incubation with FN at 4° C., the substrates were washed twice with PBS. Cells of the same concentration and volume (i.e., same number) were then seeded on each substrate.

Cell culture and adhesion. Cells of three different types were seeded on FN-coated substrates. NIH 3T3 fibroblasts were cultured in DMEM (Mediatech, Inc., Herndon, Va.) supplemented with 10% fetal bovine serum (FBS) (Hyclone, Logan, Utah). Human umbilical cord vein endothelial cells (HUVECs) were cultured with EBM-2 Basal Medium and EGM-2 SingleQuot Kit (Lonza, Walkersville, Md.). Bovine capillary endothelial cells (BCEs) were pre-cultured with low-glucose DMEM supplemented with 10% fetal calf serum (FCS) (Hyclone, Logan, Utah).

Immunostaining. After 24 hours of cell seeding, non-adherent cells were removed with two gentle washes with PBS. The samples were fixed with 4% paraformaldehyde for 20 min and washed several times with PBS. Fixed cells were immuno-stained for vinculin and stained for actin using our previously reported methods (Lele et al.; Parker et al.). Briefly, cells were fixed with 4% paraformaldehyde, permeabilized with 0.2% Triton X-100, and treated with mouse monoclonal anti-vinculin antibody (Sigma Aldrich, St. Louis, Mo.), followed by goat anti-mouse secondary antibody conjugated with Alexa Fluor 488 (Invitrogen, Eugene, Oreg.). Actin was stained with phalloidin conjugated with Alexa Fluor 594 (Invitrogen, Eugene, Oreg.). Cells were then imaged on a Nikon TE 2000 epifluorescence microscope using FITC and Texas Red filters. All images were collected using the NISElements program (Nikon).

Cell viability assay. The live/dead viability/cytotoxicity kit for mammalian cells (Invitrogen, Eugene, Oreg.) was used for quantifying adherent cell viability on each substrate. Cells were incubated at 30-45 minutes with calcein AM (2 μM for fibroblast, 5 μM for endothelial cells) and ethidium homodimer-1 (EthD-1) (4 μM for fibroblast, 1.5 μM for endothelial cells) (Michikawa et al.). Next, epifluorescence images of five random fields were collected on a Nikon TE 2000 inverted microscope using a 10× lens. The average number of cells adherent on each substrate, the number of adherent live cells (stained green with calcein AM) and adherent dead cells (stained red with EthD-1) were quantified from these images using the NIS-Elements program (Nikon). The experimental data was pooled and used for statistical comparisons using the Student's T-test.

Scanning electron microscopy (SEM). Cells were prepared for SEM by fixation with 2% glutaraldehyde buffered in PBS and post-fixed in 1% osmium tetroxide. Samples were next dehydrated in graded ethanol concentrations. Critical point drying (CPD) was performed on a Bal-Tec 030 instrument (ICBR Electron Microscopy Core Lab, University of Florida) followed by ebeam metal deposition (Ti/Au, 10/50 Å). SEM was performed on a Hitachi S-4000 FESEM (ICBR Electron Microscopy Core Lab, University of Florida). Images of samples were taken at 1.8-8.0 k× magnifications.

Time lapse imaging. Cells which had been pre-cultured as mentioned above were trypsinized and resuspended in bicarbonate-free optically clear medium containing Hank's balanced salts (Sigma Aldrich, St. Louis, Mo.), L-glutamine (2.0 mM), HEPES (20.0 mM), MEM essential and non-essential amino acids (Sigma Aldrich, St. Louis, Mo.), and 10% FCS (Lele et al.). Cells were passed onto FN-coated glass or ZnO nanorods, and phase contrast imaging performed overnight for 10 hours on the Nikon TE 2000 microscope. Images were collected every 1 minute, using a 20× objective.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1

### ZnO Nanorods Form Upright and Uniform Monolayers With the Hydrothermal Growth Method

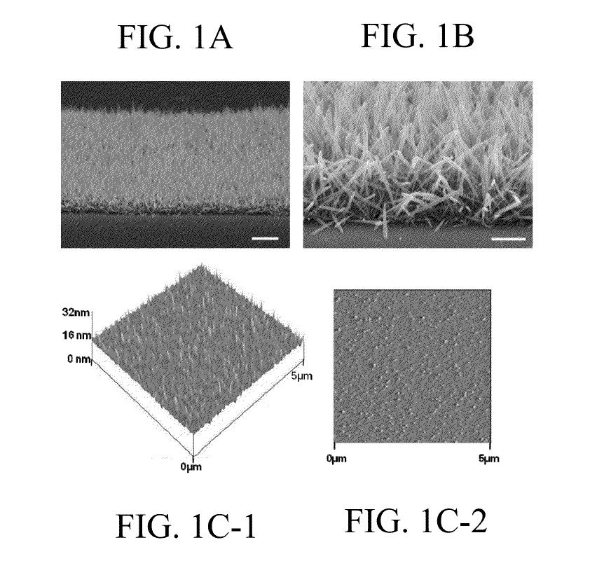

Shown in FIGS. 1A and 1B are SEM images of <001> (crystal orientation) vertically-aligned ZnO nanorod arrays of the present invention. Such nanorods could be grown over areas on the order of 1 cm²; thus ZnO nanorods could be grown in uniform monolayers over very long distances compared to cellular length scales. The nanorods of the present invention were approximately 50 nm in diameter, 500 nm in height and the density of nanorods was approximately 126 rods per square micron. Based on measured cell spreading areas, this number corresponds to approximately 60,000 nanorods per fibroblast and approximately 75,000-150,000 nanorods per endothelial cell.

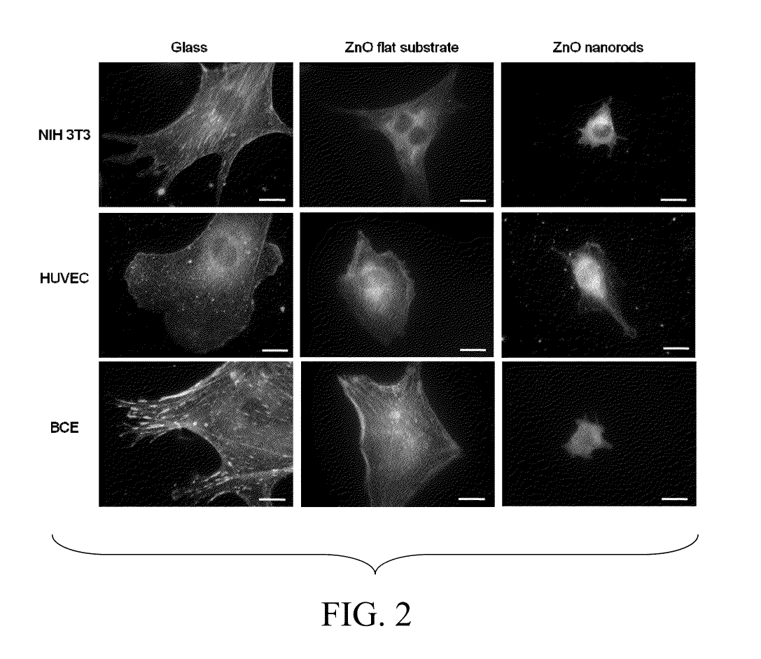

Because there was a focus on the effect of topology on cells, it was important to choose an appropriate control for statistically comparing effects of nanorods on cells. As the material itself can have effects on protein adsorption and cell adhesion, a topologically smooth substrate made of ZnO—a thin film commercially available from Cermet Inc.—was chosen. AFM images of this substrate are shown in FIGS. 1C-1 and 1C-2. The flat substrate is smooth over long length scales, with an average roughness of 1.33 nm. Interestingly, similar results were obtained for glass (average roughness of 1.34 nm, not shown), which allowed for comparison of the performance of the ZnO flat substrate and ZnO nanorods with glass, a well-established substrate for cell culture.

### Example 2

### Cells on ZnO Nanorods Show Decreased Spreading and Focal Adhesion Formation

The influence of ZnO nanorods of the present invention on cell spreading was also investigated. Cells in vitro spread by assembling focal adhesions and stress fibers.

FIG. 2 shows fluorescence images of three different cell types—NIH 3T3s, HUVECs, and BCEs on glass, ZnO flat substrate, and ZnO nanorods. Cells on ZnO flat substrates and glass cover slips assembled clear focal adhesions and stress fibers. Focal adhesions and stress fibers were not visible in cells on nanorods. The average area of cell spreading was decreased significantly on nanorods compared with ZnO flat substrates (a reduction of 60-70%, see Table 1). These trends were observed in each of the three cell types.

### Example 3

### Cell Number and Viability are Decreased on Nanorods

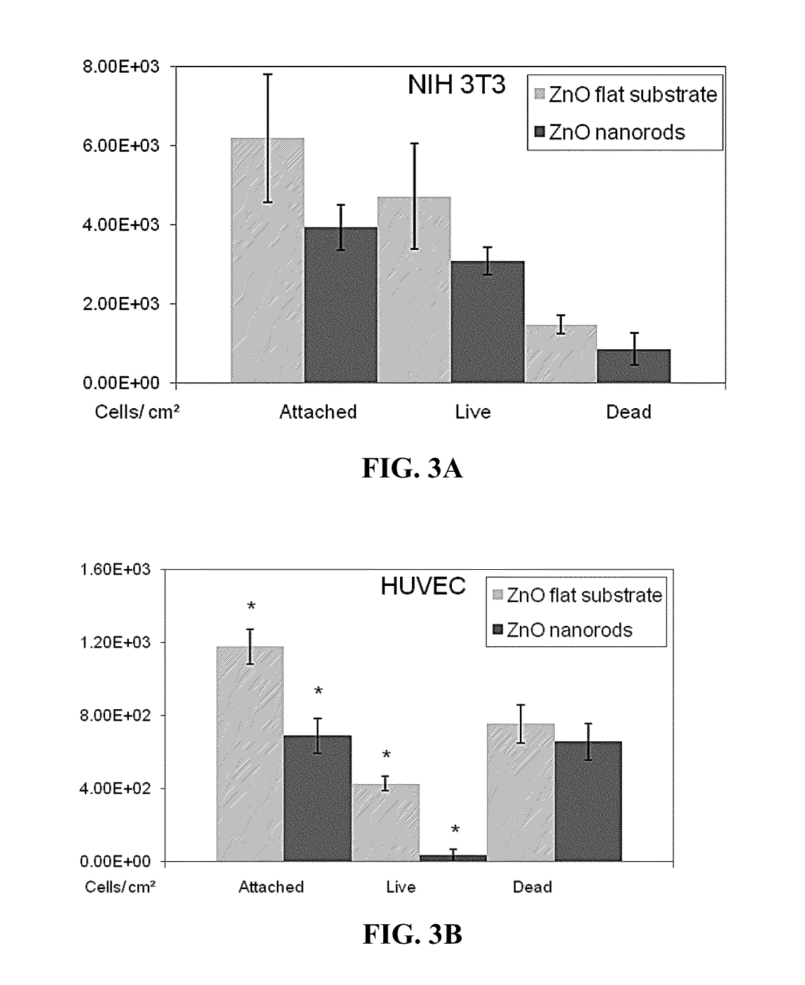

The results of Kim et al. (2007a) suggest that mesenchymal stem cells can survive on silicon nanowires for several days. Cells in Kim et al. (2007a) were only exposed to 20-30 nanowires per cell. To investigate if a confluent monolayer of ZnO nanorods of the present invention supports cell survival, the total cell number, and fraction of live and dead cells in an adherent population at 24 hours of culture was quantified. The total number of adherent cells and of adherent live cells at the end of 24 hours was greatly decreased on nanorods of the present invention compared to flat substrates (FIGS. 3A-3D). Because cells were seeded at equal cell densities on the two substrate types, the ratio of the number of live cells on the nanorods represents the effect of topography (free from any other effects) on cell survival (FIG. 3D). There was an order of magnitude decrease in cell survival in endothelial cells, and cell survival decreased by ˜40% in fibroblasts (FIG. 3D). The fact that the fraction of attached live cells decreased on the nanorods in all three cell-types, are consistent with previous observations that topological cues at the nano-scale can profoundly modulate cell behavior (Choi et al.; Gonsalves et al.; Girard et al.).

### Example 4

### Cells Cannot Assemble Lamellipodia on Nanorods

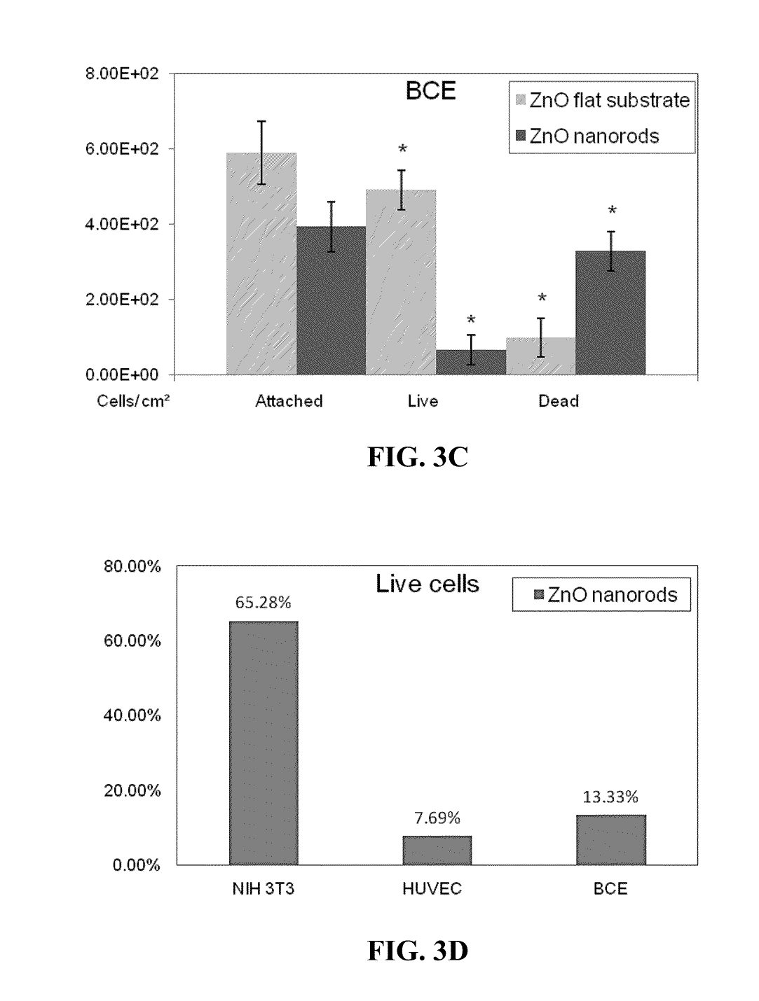

A recent study showed that cells on needle-like nanostructures only assemble filopodia (Choi et al.). To investigate this possibility for ZnO nanorods, SEM studies on NIH 3T3 fibroblasts cultured on ZnO nanorods of the present invention were performed (FIGS. 4A-4D). Most cells on ZnO nanorods were rounded (FIGS. 4A and 4B). Instead of flat sheet-like lamellipodia, some cells formed thin processes (black arrow in FIG. 4D) and thin filopodia-like structures (white arrows in FIG. 4D) that appeared to attach to the ZnO nanorods. Therefore, while cells can attach to the ZnO nanorods of the present invention using filopodia-like structures, they are not able to spread on the nanorods.

### Example 5

### Initial Cell Spreading is Abrogated on Nanorods

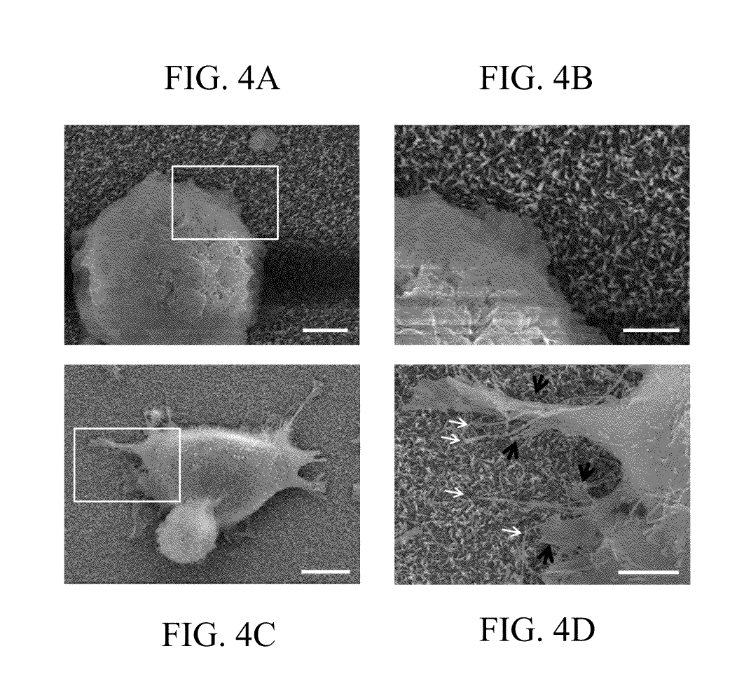

The results of Kim and co-workers showed that Si nanowires with diameter similar to our nanorods are engulfed by cells (Kim et al., 2007a). This raises the possibility that cells may spread initially on the nanorods but undergo apoptosis due to engulfment of nanorods at longer times. To clarify this, time-lapse imaging was performed for studying dynamic cell spreading on nanorods (FIGS. 5A and 5B). After seeding, initial adhesion of HUVECs on glass occurred in the first hour (FIG. 5A). Lamellipodia formation could be seen from 2 hours onward followed by complete spreading at approximately 5 hours (white arrows in FIG. 5A). Conversely, on nanorods of the present invention, little initial spreading occurred and cells remained rounded over several hours (FIG. 5B). No lamellipodia formation was visible. These results show that nanorods did not support initial cell spreading. While these results alone do not rule out long-term toxicity of nanorods due to engulfment, they provide evidence that cells are not able to initially spread on nanorods, which may contribute to decreased survival at long times.

### MATERIALS AND METHODS FOR EXAMPLES 6-10

Fabrication of ZnO nanorods. ZnO nanorods were made by a solution-based hydrothermal growth method (Lee et al., 2008). First, ZnO nanoparticles were prepared by mixing 10 mM zinc acetate dehydrate (Sigma Aldrich, St. Louis, Mo.) with 30 mM of NaOH (Sigma Aldrich, St. Louis, Mo.) at 58° C. for 2 h. Next, ZnO nanoparticles were spin-coated onto the substrate several times and then post-baked on a hot plate at 150° C. to promote adhesion. Seeded substrates were then suspended upside down in a Pyrex glass dish filled with an aqueous nutrient solution. The growth rate was approximately 1 μm/h with 100 ml aqueous solution containing 20 mM zinc nitrate hexahydrate and 20 mM hexamethylenetriamine (Sigma Aldrich, St. Louis, Mo.). To arrest the nanorod growth, the substrates were removed from solution, rinsed with de-ionized water and dried in air at room temperature. Zinc oxide was deposited for relatively “flat” control samples of sputtered ZnO using a Kurt Lesker CMS-18 Multi Target Sputter Deposition system.

Macrophage generation. Bone marrow-derived macrophages were generated from 7-10 week-old C57BL6/J mice using a 10 day culture protocol. Animals were handled in accordance with protocol approved by the University of Florida. Briefly, mice were euthanized by CO₂ asphyxiation followed by cervical dislocation and tibias and femurs were harvested for isolating marrow cells. The marrow cells were obtained by flushing the shaft of the bones with a 25 gauge needle using RPMI (Hyclone Laboratories Inc, Logan, Utah) medium containing 1% FBS (Hyclone Laboratories Inc, Logan, Utah) and 1% PSN (Hyclone Laboratories Inc, Logan, Utah). The red blood cells (RBCs) were removed by lysing with ACK lysis buffer (Lonza, Walkersville, Md.) followed by plating on tissue culture flasks for 2 days in order to remove adherent cells. After 48 hours (day 2), the floating cells were transferred to low attachment plates and cultured with 1 ng/ml IL-3 for expansion of the macrophage precursor cells. The cells were cultured in macrophage culture media consisting of Dulbecco's Modified Eagle's Medium (DMEM)/F12(1:1) (Cellgro, Herndon, Va.) medium containing 1% PSN, 1% L-glutamine (Lonza, Walkersville, Md.), 1% Non essential amino acids (NEAA) (Lonza, Walkersville, Md.), 1% Sodium pyruvate (Lonza, Walkersville, Md.), 10% Fetal Bovine Serum (FBS) and 10% L-929 cell conditioned media. (LCCM). To produce LCCM, L-929 cells were grown to a confluent monolayer in 150 cm² tissue culture flasks. 50 ml media was added to each flask for 7 days after which all the media in the flask was replaced with fresh media for 7 additional days. The media collected at day 7 and 14 was pooled, sterile filtered and stored at −20° C. The LCCM serves as a source of Macrophage Colony Stimulating Factor (MCSF) which pushes the differentiation of marrow cells towards the macrophage phenotype. Half of the media in the wells was exchanged on day 4 with fresh macrophage culture media. At day 6, cells from low attachment plates were transferred to tissue culture 6 well plates to allow macrophage adhesion and maturation. At day 8 all the media in the wells was replaced with fresh media and at day 10 of culture the cells are ready for experiment. The purity of the macrophage culture was verified by staining for CD11b and F4/80 murine macrophage markers and analyzed using flow cytometry.

Preparation of substrates for macrophage culture. The ZnO nanorods were grown on 22 mm square glass coverslips. 22 mm square glass coverslips (Fisherbrand, Fisher scientific) were used as reference substrates and sputtered ZnO “flat” substrate served as a control for surface topography. Prior to cell seeding the ZnO substrates-nanorod and sputtered, were sterilized by alcohol wash for 15 min followed by UV treatment for 15 min. The glass coverslips were O₂ plasma etched followed by alcohol wash and UV treatment.

Macrophage culture on ZnO substrates. In order to study adhesion of macrophages on nanorods over several hours, macrophages were cultured overnight on ZnO nanorods. Phase contrast imaging was performed overnight for 10 h on the Nikon TE 2000 microscope. Images were collected every 1 min, using a 40× objective.

Adhesion and viability studies were performed using macrophages pre-loaded with calcein (Keselowsky et al., 2003). Briefly, macrophages were loaded 1 μg/ml calcein-AM (AnaSpec Inc, San Jose, Calif.) in 2 mM dextrose solution by incubating it at 37° C. for 20 min, pelleted and resuspended in macrophage culture media. Macrophages in this cell suspension were counted by hemocytometer and 500,000 cells were seeded onto substrates for 16 hours. The number of cells adherent on the surfaces was quantified at 3 and 16 hours post seeding. At the time of quantification, 7-AAD (Beckman Coulter, Fullerton, Calif.) was added to the wells as per manufacturer's instruction. Adherent cells that retained calcein and did not stain with 7-AAD were counted as live while 7-AAD positive cells were counted as dead. Three images were taken per sample for quantifying the total adherent, live and dead cells. Data was averaged from 6-8 replicates obtained from 3 separate runs.

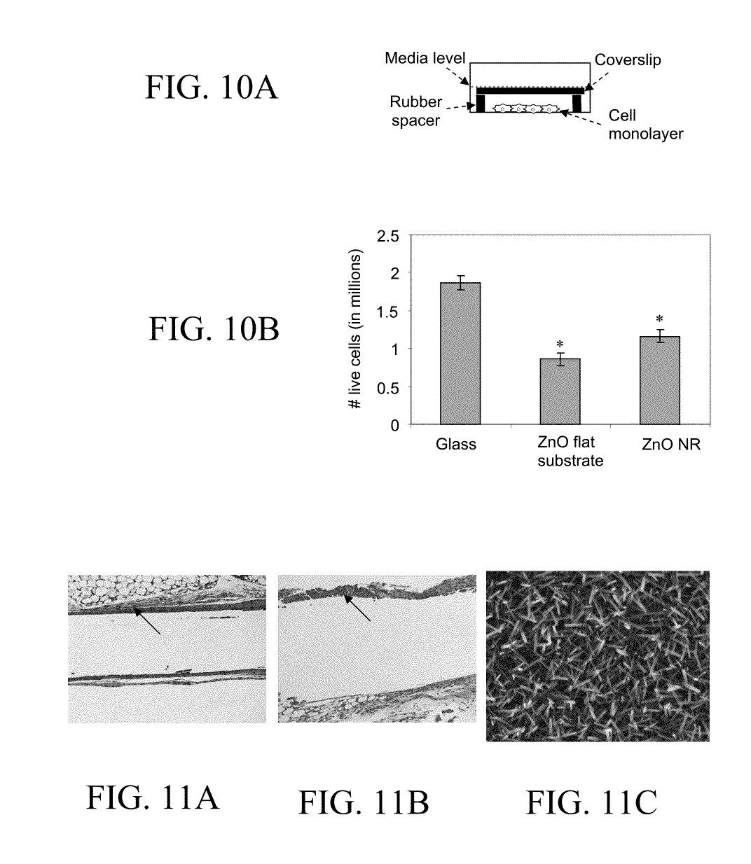

In order to test the non-contact based cytotoxicity of ZnO substrates, an equal number of macrophages in 6 well plates were exposed to the three surfaces inverted on Viton O-rings (FIG. 10A). The surfaces were maintained in the inverted position in the wells for 7 days with media change every alternate day. At day 7, the substrates were removed and 7-AAD was added to the wells in order to stain the dead cells. Phase contrast images of 3 fields per well were taken in order to quantify the number of viable cells adherent on the wells. The data was collected and averaged from 8 replicates obtained from 2 separate runs.

In vivo response to ZnO nanorod coating. As a pilot study of the foreign body response mounted by the body against ZnO nanorod coated biomaterials, polyethylene terephthalate (PET) discs coated with ZnO nanorods were implanted subcutaneously on the mouse's back on the dorsal side of the thorax. Uncoated PET discs served as a control. Due to the toxic effect of ZnO NR on macrophages observed during the in vitro studies we conducted a pilot run with 2 NR coated discs implanted in a mouse. Xx mice served as controls.

2 discs (7 mm diameter, 0.5 mm thick) were cut from PET sheets and ZnO nanorods were grown on its surface as described in section 2.1. Prior to implantation, the nanorod coated and uncoated discs were sterilized by dipping in 70% ethanol. 2 discs were implanted subcutaneously on the mouse's back in accordance with protocol approved by the University of Florida IACUC committee. The wounds were closed with wound clips which were removed at day 7 after implantation. PET discs were explanted at 14 days, formalin-fixed and paraffin-embedded. Histological sections (5 μm thick) were stained with hemotoxylin and eosin stain for nuclei (dark blue) and collagen (pink) and examined by phase-contrast microscopy.

Statistical Analysis. Statistical analyses were performed using general linear nested model ANOVA, linear regression analysis using Systat (Version 12, Systat Software, Inc., San Jose, Calif.). Pair-wise comparisons were made using Tukey's Honestly-Significant-Difference Test with p-values of less than or equal to 0.05 considered to be significant.

### Example 6

### Macrophage Culture and Purity

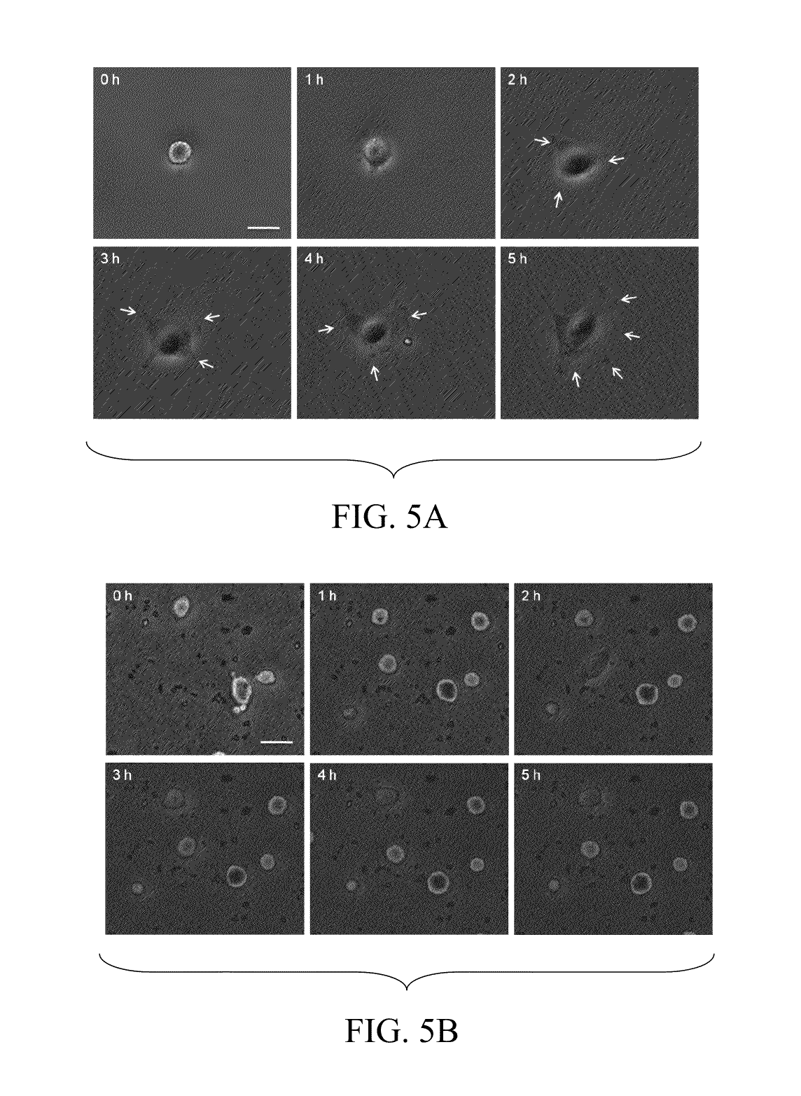

In order to generate a large number of highly purified macrophages, we used a modified 10 day culture protocol (Cunnick et al., 2006; Stanley, 1997) for generating macrophages from bone marrow precursor cells. In order to confirm macrophage culture purity, we determined the percentage of cells that matured to macrophages by staining with CD11b and F4/80, murine macrophages markers and analyzed using flow cytometry. The percentage of cells co-expressing CD11b and F4/80 was ˜90% (FIG. 6A) indicating the ability to generate a highly pure macrophage population.

### Example 7

### Macrophage Spreading is Inhibited on ZnO Nanorods

In order to investigate effect of surface topology on macrophage spreading and adhesion we studied macrophage spreading on ZnO nanorods over several hours post seedin. The ZnO nanorod surface was coated with fibronectin by overnight incubation followed by PBS wash. Macrophages were seeded on the nanorod substrate and allowed to adhere for 1 hour after which a time lapse video of a single cell was taken in order to study the change in spread area of the cell over time. Initially the cell was well spread for the first 2 hours however over the next couple of hours its area of cell spreading was decreased significantly. Due to the unfavorable surface topology of the nanorods, macrophages may fail to assemble focal complexes required for cell adhesion and spreading.

### Example 8

### Decrease in Macrophage Viability in Contact With ZnO

Several reports have suggested that nanotopography controls cell adhesion and viability for example Lee et al have reported decreased adhesion and viability of endothelial cells on ZnO nanorods as compared to ZnO flat substrate (Lee et al., 2008). In order to determine whether the nanotopography of ZnO nanorods was able to modulate macrophage adhesion, we first investigated contact-dependent response of macrophages to ZnO nanorod surface as compared to sputtered ZnO substrate (as a relatively “flat” ZnO surface) and O₂-plasma etched cleaned glass coverslips as a reference. To determine the viability of macrophages in contact with substrates, equal numbers of calcein-loaded macrophages were plated on the 3 substrates for 16 hours. Cells maintaining calcein loading and 7-AAD exclusion were counted as live while the ones that either retained or released calcein but stained for 7-AAD were counted as dead. The sum of the live and dead cells was computed for total adherent cell number.

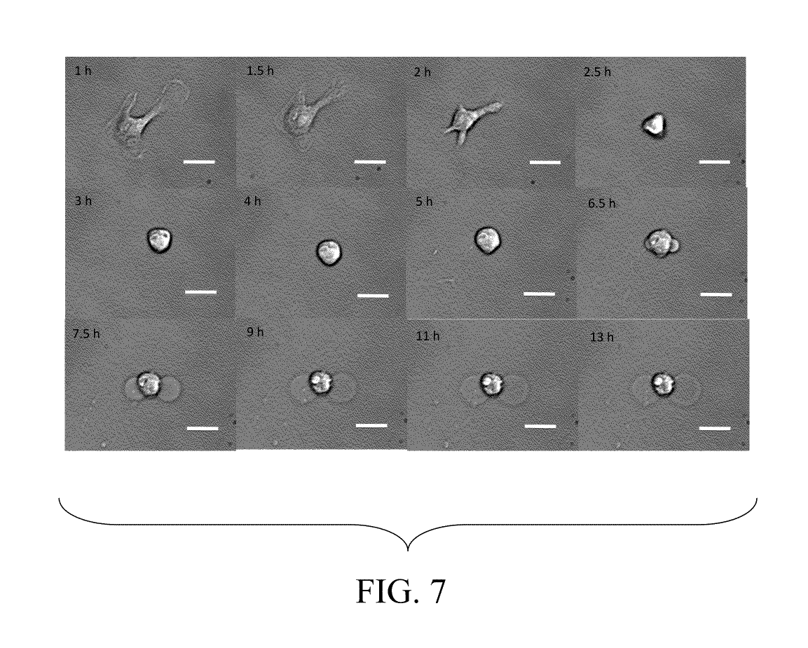

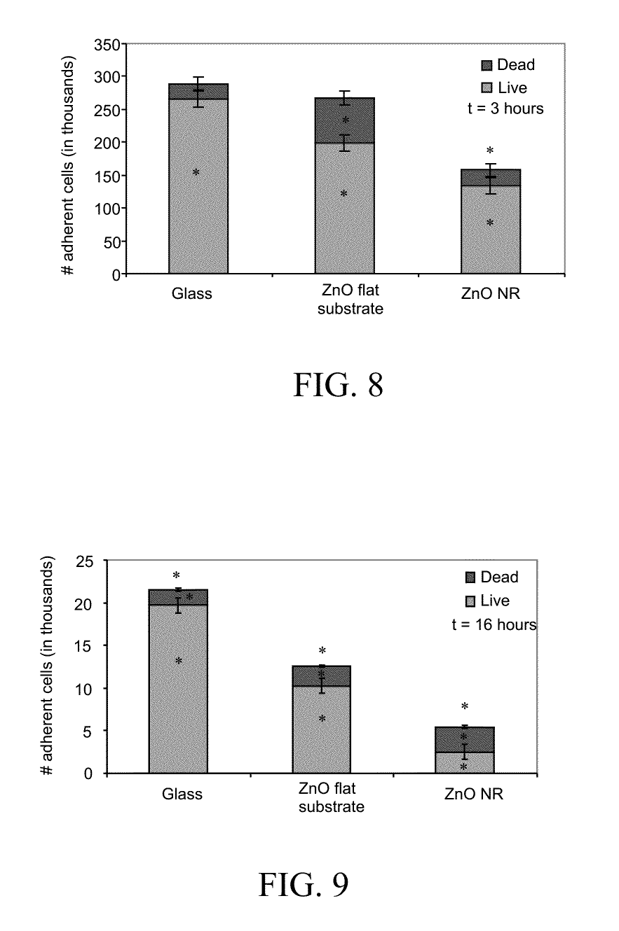

At both 3 and 16 hours, the total number of live cells adherent on the ZnO flat substrate and ZnO nanorods was greatly reduced as compared to the reference glass surface (FIGS. 8 and 9). At 3 hours the number of live cells adherent on sputtered ZnO and ZnO nanorods was 75% and 50% of the live cells adherent on glass, respectively.

At 3 hours the total number of cells adherent on glass and ZnO flat substrate was not significantly different, however the number of dead cells was significantly higher (3× compared to glass) on ZnO substrate (FIG. 8). The number of dead cells on the ZnO nanorods was almost equal to glass although the number of cells adherent on nanorod surface was significantly lower (FIG. 8) thus indicating that the cells either failed to adhere to nanorod surface or adhered but died and hence floated off into the media. At 16 hours the total number of dead cells adherent on the ZnO flat substrate and ZnO nanorods was greater as compared to the reference glass surface suggesting an intrinsic toxicity of ZnO to macrophages (FIG. 9). The number of dead cells adherent on sputtered ZnO and ZnO nanorods were 1.3× and 1.6× times the number of dead cells adherent on glass. At 16 hours the number of live cells adherent on sputtered ZnO and ZnO nanorods was 52 and 12% of the live cells adherent on glass. Additionally the number of cells adherent and viable of ZnO nanorods was significantly lower as compared to ZnO flat substrate indicating a role of surface topology in cytotoxicity (FIG. 9). The toxicity can be attributed to various macrophage ZnO interactions for example the ZnO from the sputtered zinc and nanorods may dissolve in the macrophage media and get internalized by macrophages inducing toxicity. Additionally a large number (˜60,000-70,000) of nanorods contact a single cell which may pierce the cell membrane leading to a loss of membrane integrity result in death.

### Example 9

### Non Contact Based Cytotoxicity of ZnO

As indicated by our results of contact based toxicity of ZnO, both sputtered ZnO and nanorods have a cytotoxic effect on the macrophages. One of the possible mechanisms by which ZnO can induce cytotoxicity is by dissolving into the cell media and being internalization by macrophages. In order to test this hypothesis of ZnO dissolution in cell media, we incubated the macrophages in media with the substrates inverted at media level as shown in FIG. 10A. All wells were plated with the same cell density and incubated with the inverted substrates for 7 days with media change every alternate day. At the end of 7 days, the percentage of viable cells was determined by counting cells from bright-field microscopy images and eliminating cells stained with 7-AAD from the count. Due to the media change every alternate day the dead cells were washed away leaving behind the adherent live cells hence the number of cells stained with 7-AAD at 7 days was negligible and not included in analysis. The number of cells viable after 7 days of culture with media in contact with ZnO flat substrate and ZnO nanorods was 45% and 62% as compared to inverted glass coverslip (FIG. 10B). The decreased viability of macrophages for ZnO flat substrate as compared to nanorods indicates that there may be greater dissolution of ZnO from sputtered zinc as compared to nanorods as ZnO has an amorphous structure, whereas the nanorods have directional growth.

### Example 10

### Foreign Body Response to Zinc Nanorod Coated Biomaterial

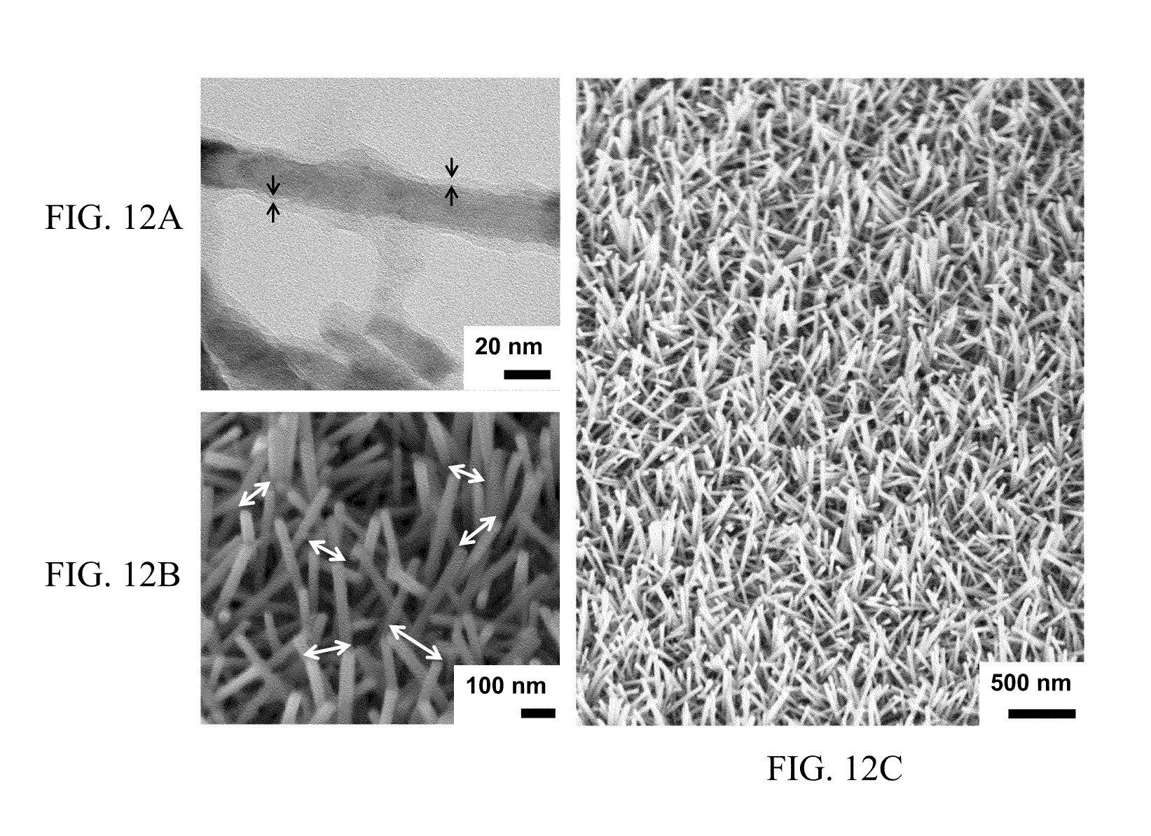

Although our in vitro data provides a valuable insight into the cytotoxic effect of ZnO surfaces and particularly nanorods on macrophages, it cannot completely represent the response of the body to a material implanted in vivo. In order to study tissue responses to ZnO coated biomaterials which are more clinically relevant; we performed a pilot study of subcutaneous implantation of ZnO nanorod coated PET discs. One of the standard methods to evaluate chronic inflammation to synthetic materials is measurement of fibrous capsule thickness following subcutaneous implantation (Anderson, 2001). PET discs coated with ZnO nanorods were implanted subcutaneously for 14 days to assess the effect of the ZnO nanorod coating on the foreign body reaction and fibrous encapsulation of implanted materials. PET discs without any coating were implanted as controls. After 2 weeks of implantation, the uncoated PET discs implanted subcutaneously on the mice's back had a smooth acellular fibrous capsule (FIG. 11A) which forms as part of the foreign body response that the body mounts against implanted foreign materials. However when the PET discs were coated with ZnO nanorods there was no formation of the smooth fibrous capsule, instead there was an accumulation of cells most probably macrophages around the disc (FIG. 11B). There are 3 stages of inflammatory response mounted by the body against biomaterials. The first stage which is the acute response comprises mainly of neutrophils. The second stage which is the chronic stage is mounted mainly by macrophages and foreign body giant cells which form a granulation tissue around the implant. The 3^{rd} stage of which is resolution of the inflammation is characterized by infiltration of fibroblasts and formation of smooth fibrous capsule around the implant. The discs coated with ZnO nanorods remain in the 2^{nd} stage of chronic inflammation failing to form a fibrous capsule indicating that the ZnO nanorods had a toxic effect on the cells involved in the 2^{nd} stage of inflammation. As seen in our in vitro results, ZnO is toxic to macrophages both in contact as well as when present in the same media and this same effect is confirmed with our in vivo disc implant data and hence does not warrant further implantation experiments.

### MATERIALS AND METHODS FOR EXAMPLES 11-14

Fabrication of nanorods. ZnO nanorods were made by a solution-based hydrothermal growth method (Kang et al., 2007). First, ZnO nanocrystal seed solutions were prepared by mixing 15 mM zinc acetate dihydrate (Sigma Aldrich, St. Louis, Mo.) with 30 mM of NaOH (Sigma Aldrich, St. Louis, Mo.) at 60° C. for 2 h. Next, ZnO nanocrystals were spin-coated onto the substrate and then post-baked on a hot plate at 200° C. for better adhesion. The substrate with these seeds was then suspended upside down in a Pyrex glass dish filled with an aqueous nutrient solution. The growth rate was approximately 1 μm per hour with 100 ml aqueous solution containing 20 mM zinc nitrate hexahydrate and 20 mM hexamethylenetriamine (Sigma Aldrich, St. Louis, Mo.). To arrest the nanorod growth, the substrates were removed from solution, rinsed with de-ionized water and dried in air at room temperature. SiO₂ was deposited with a Unaxis 790 plasma enhanced chemical vapor deposition (PECVD) system at 50° C. using N₂O and 2% SiH₄ balanced by nitrogen as the precursors as reported before (Chu et al., 2008). Patterned nanorods were fabricated by conventional photoresist (PR) lithography (Kang et al., 2007). A glass coverslide was processed with negative PR (SU-8 2007, Microchem) so that a pattern with 50 micron circles was formed on the surface. The substrate was then post-baked at 110 C.° for 30 min. The processed substrate was spin-coated with ZnO nanocrystals as seed materials and nanorods were grown on the substrate with an aqueous nutrient solution. The negative PR was removed by PG remover in a warm bath at 60 C.° for 30 min.

Cell culture. For control substrate, we used 22 mm square glass cover slips (Corning, Inc., Lowell, Mass.). Before use, each substrate was sterilized with UV for 5 min and cleaned in 70% ethanol and de-ionized water. After drying substrates in air at room temperature, they were treated with 5 μg/ml human fibronectin (FN) (BD biosciences, Bedford, Mass.). After overnight incubation with FN at 4° C., the substrates were washed twice with PBS. NIH 3T3 fibroblasts were cultured in DMEM (Mediatech, Inc., Herndon, Va.) supplemented with 10% donor bovine serum (DBS) (Hyclone, Logan, Utah). Human umbilical cord vein endothelial cells (HUVECs) were cultured in EBM-2 Basal Medium and EGM-2 Single Quot Kit (Lonza, Walkersville, Md.). Cell suspensions of the same concentration and volume (i.e. same number of cells) were then seeded on each substrate.

Immunostaining and cell viability assay. After 24 hours of cell seeding, non-adherent cells were removed with two gentle washes with PBS. The samples were fixed with 4% paraformaldehyde for 20 min and washed several times with PBS. Fixed cells were immuno-stained for vinculin and stained for actin and nucleus using our previously reported methods (Lee et al., 2008). Briefly, cells were fixed with 4% paraformaldehyde, permeabilized with 0.2% Triton X-100, and treated with mouse monoclonal anti-vinculin antibody (Sigma Aldrich, St. Louis, Mo.), followed by goat anti-mouse secondary antibody conjugated with Alexa Fluor 488 (Invitrogen, Eugene, Oreg.). Actin was stained with phalloidin conjugated with Alexa Fluor 594 (Invitrogen, Eugene, Oreg.) and nucleus was stained with 4′-6-diamidino-2-phenylindole (DAPI) (Sigma Aldrich, St. Louis, Mo.). Cells were then imaged on a Nikon TE 2000 epifluorescence microscope using GFP, Texas Red and DAPI filters. All images were collected using the NIS-Elements program (Nikon).

The live/dead viability/cytotoxicity kit for mammalian cells (Invitrogen, Eugene, Oreg.) was used for quantifying adherent cell viability on each substrate. Cells were incubated at 30-45 minutes with calcein AM (2 μM for fibroblast and 4 μM for endothelial cells) and ethidium homodimer-1 (EthD-1) (4 μM for all types of the cells). Next, epifluorescence images of six to ten random fields were collected on a Nikon TE 2000 inverted microscope using a 10× lens for NIH 3T3 and HUVEC. The average number of cells adherent on each substrate, the number of adherent live cells (stained green with calcein AM) and adherent dead cells (stained red with EthD-1) were quantified from these images using the NIS-Elements program (Nikon). Three independent experiments of cell viability were performed and the data were pooled. The average area of cell spreading was determined from three independent experiments with statistical comparison using the Student's T-test.

Time lapse imaging. Cells were pre-cultured on the patterned nanorods for 24 hours as mentioned above. Before taking a movie, non-adherent cells were removed with two gentle washes with PBS and new media was added to the dish. Phase contrast imaging was performed for 6 hours on the Nikon TE 2000 microscope with humidified incubator (In Vivo Scientific, St. Louis, Mo.). Images were collected every 5 minutes using a 10× objective.

### Example 11

### Fabrication of SiO₂ Coated Nanorods

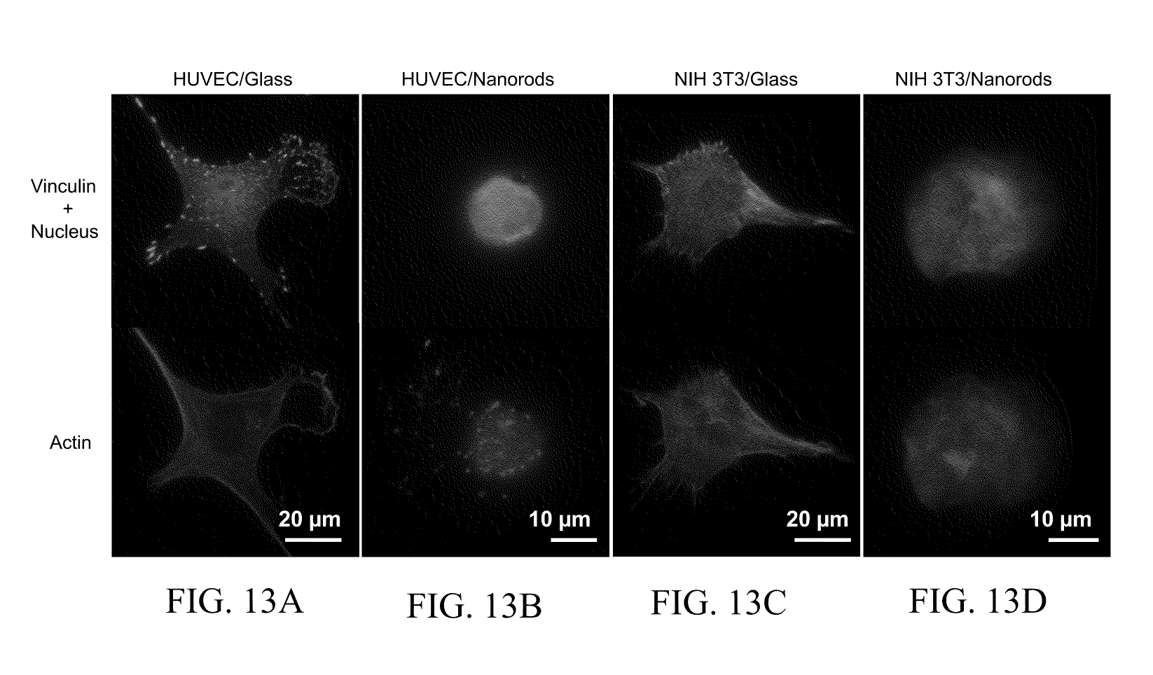

Many biomedical implants are made of temperature-sensitive materials such as plastic. Hence, it is necessary to grow nanorods with techniques that do not require high temperature. Densely packed ZnO nanorods were fabricated with a low-temperature (95° C.) hydrothermal, solution-based growth method (Kang et al., 2007). We next deposited nano-thin films of SiO₂ with controlled thickness, 50 Å, using PECVD at 50° C. according to our previously published methods (Chu et al., 2008). Transmission electron microscopy (TEM) images of the resulting nanorods with 50 Å thickness of SiO₂ nano-films deposited are shown in FIG. 12A. The nanorods were randomly oriented in the upright direction, approximately 40˜50 nm in diameter, 500 nm in height. The average spacing between nanorods was approximately 80 to 100 nm (FIG. 12B, white arrows). Importantly, the SiO₂ coatings were deposited uniformly on each nanorod free of any local defects, which was confirmed with TEM, local electrical conductance measurements, chemical wet-etching and photoluminescence intensity measurements (Chu et al., 2008). Our technique thus resulted in randomly oriented, upright SiO₂ deposited nanorods that cover the surface with densely packed monolayers without any defects over cm length scales (FIG. 12C).

### Example 12

### Decreased Cell Adhesion on SiO₂ Coated Nanorods

Cell adhesion and spreading occurs by the ligation of trans-membrane integrins to ligands (such as fibronectin) immobilized on the surface. This is followed by clustering of the integrins at the nanoscale, and subsequent formation of multi-protein, micron-scale assemblies called focal adhesions (Bershadsky et al., 2006). Focal adhesions allow force transfer from the contractile acto-myosin cytoskeleton inside the cell to the outside surface, and this allows cells to adhere to and spread on the surface. If focal adhesions are not allowed to assemble in cells that depend on anchorage for survival, this leads to weak attachment to the surface, lack of cell spreading and subsequent apoptosis (Chen et al., 1997; Re et al., 1994). Therefore, the assembly of focal adhesions was next studied using immunofluorescence microscopy.

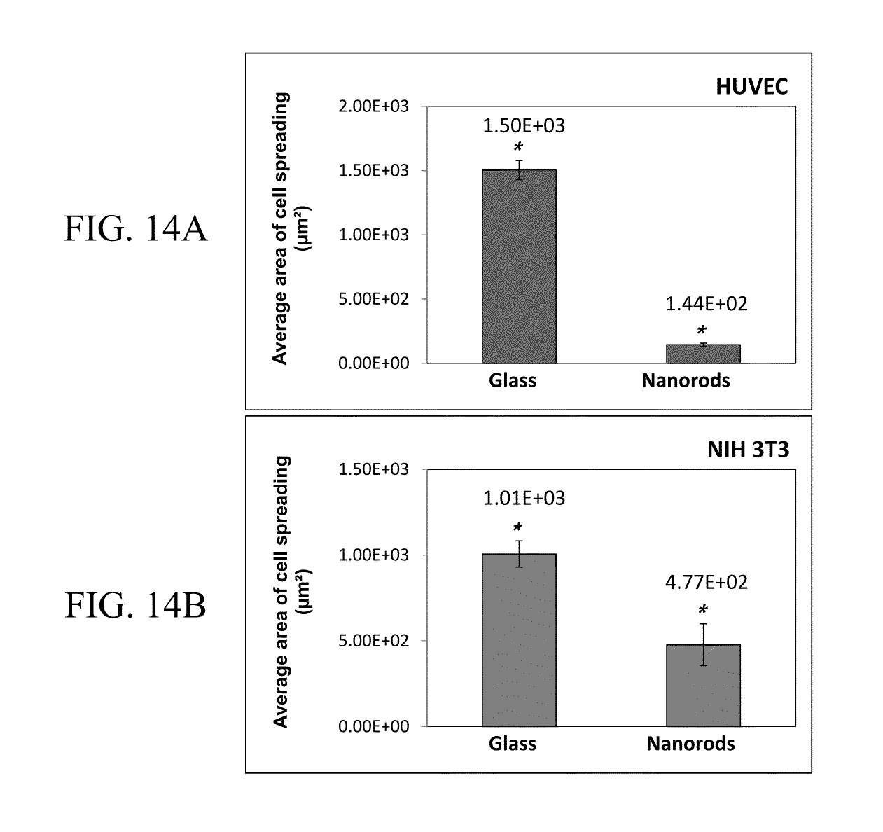

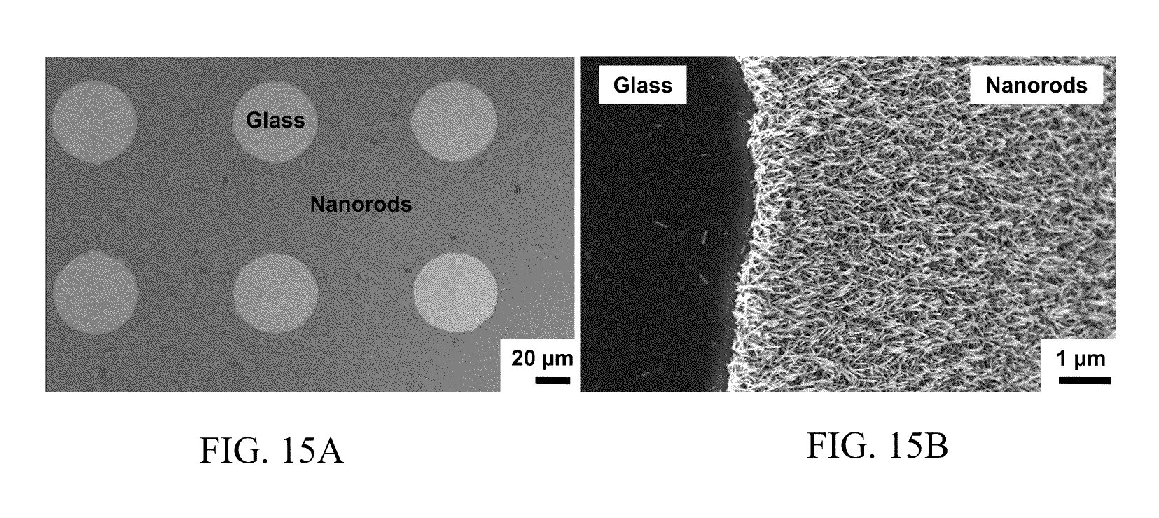

Human umbilical vein endothelial cells (HUVECs) and NIH 3T3 fibroblasts were cultured on SiO₂ nanorods which were pre-incubated with fibronectin overnight. Cells were fixed with paraformaldehyde and stained for vinculin, actin stress fibers and the nucleus. Both HUVECs and NIH 3T3 fibroblasts assembled vinculin-labeled focal adhesions on glass (FIGS. 13A and 13C). On the nanorod-coated surfaces, focal adhesions were not visible and cells were rounded and poorly spread (FIGS. 13B and 13D). Cells on nanorods were also unable to assemble contractile stress fibers. Consequently, the average area of cell spreading on nanorods was significantly decreased (FIGS. 14A and 14B) with a lack of focal adhesion and stress fiber formation. This result suggests that cells are unable to spread and assemble focal adhesions on nanorods, which may cause apoptosis in these adhesion-dependent cells (Chen et al., 1997; Re et al., 1994).

### Example 13

### Cell Adhesion Patterning With Patterned Nanorods

Recent work by Spatz and co-workers showed that focal adhesion assembly requires the spacing between ligated integrins to be less than 70 nm (Arnold et al., 2004; Girard et al., 2007). A spacing of more than 73 nm between ligated integrins limits attachment, spreading, and actin stress fiber formation in fibroblasts. As the diameter of the SiO₂ nanorods is approximately 40˜50 nm, local integrin clustering may occur but to a very limited extent given the vertical nature and small length (500 nm) of the nanorods. Due to the spacing of 80˜100 nm, integrin clustering may not occur over multiple nanorods, preventing the assembly of contiguous focal adhesions on the micron length scale (FIGS. 13B and 13D).

Other possible explanations for the reason that cells cannot spread on nanorods is the anti-wetting nature of nanostructured surfaces (Sun et al., 2005). However, cells do not spread like passive droplets, but through actin polymerization which is determined by intracellular processes (Dickinson et al., 2004) as long as initial adhesion is possible (Bershadsky et al., 2006). In addition, Kim et al. (Kim et al., 2007a) have observed that cells are easily able to engulf SiO₂ nanowires with similar diameter as that studied here. This indicates that local cell-membrane interactions with nanorods are not impeded by alterations in protein adsorption.

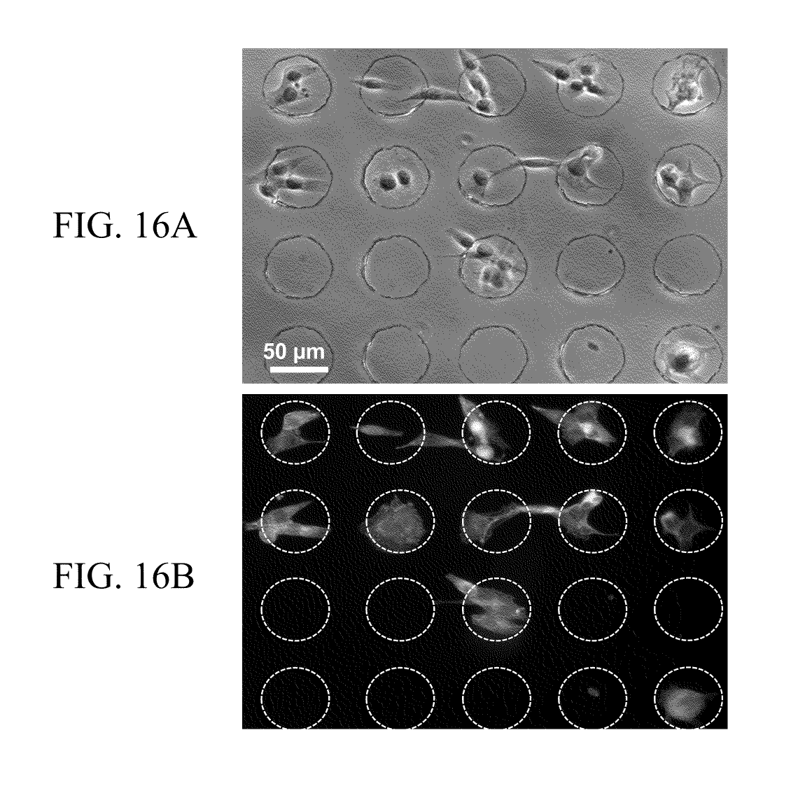

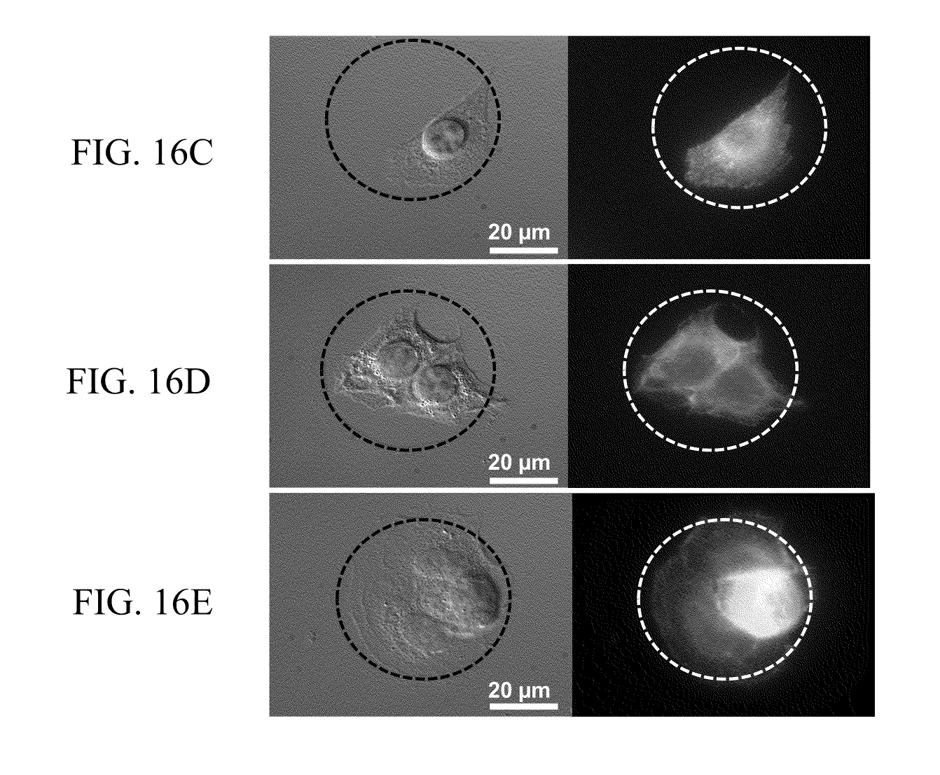

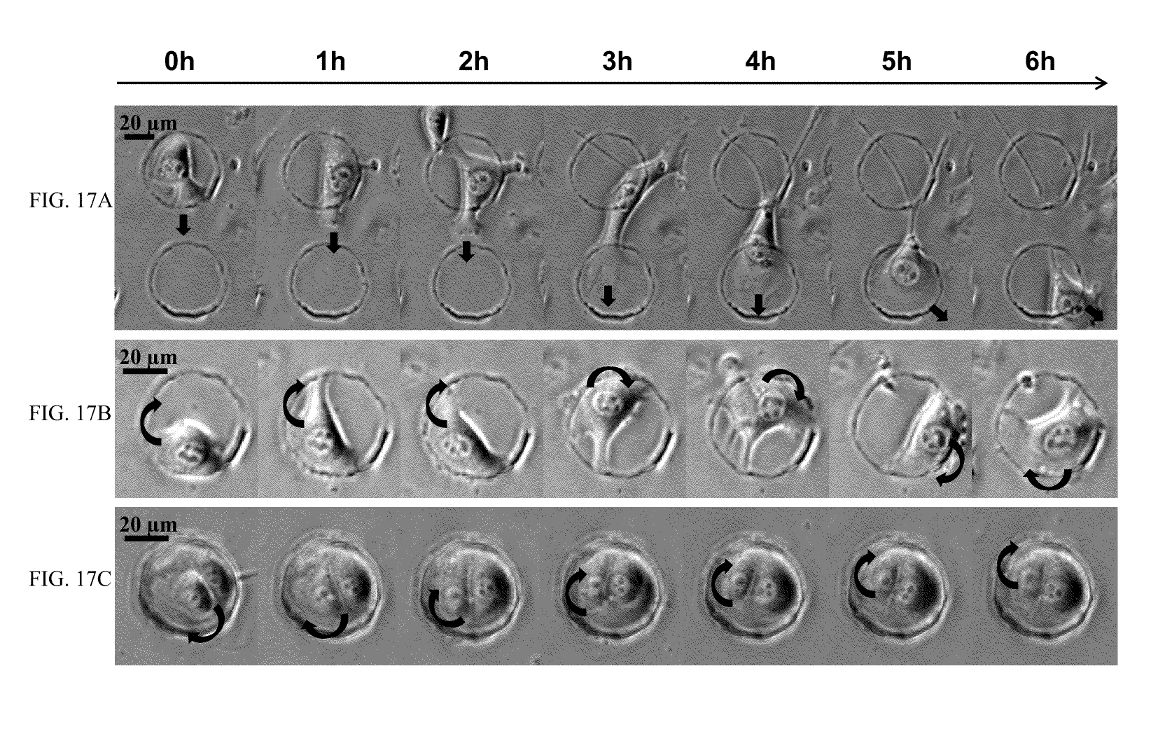

To further investigate the question of de-wetting, we spatially patterned nanorods using a low-temperature, and patterned growth method (Kang et al., 2007). This method results in patterned nanorods that are not present inside circles, and are present outside in dense monolayers (FIGS. 15A and 15B). The diameter of circles was 50 μm and spacing between the circles was 40˜60 μm. Nanorods were 50 nm in diameter and 500 nm in height. FIGS. 16A-16E show that fibroblasts preferably adhered to the flat surface rather than to the nanorods after 48 hour culture. While the cells were confined to the circular regions on average, cells were frequently able to migrate from circle to circle by spanning the intervening nanorods (FIGS. 17A-17C). As the cell is able to migrate from one flat area to another, by weakly adhering to the intervening nanorods, de-wetting does not play a significant role in determining cell interactions with nanorods (Sun et al., 2005). Interestingly, this result suggests that the patterned nanorods provide a new way of dynamically patterning cells and therefore creating complex tissues.

### Example 14

### Decreased Cell Survival on Nanorods

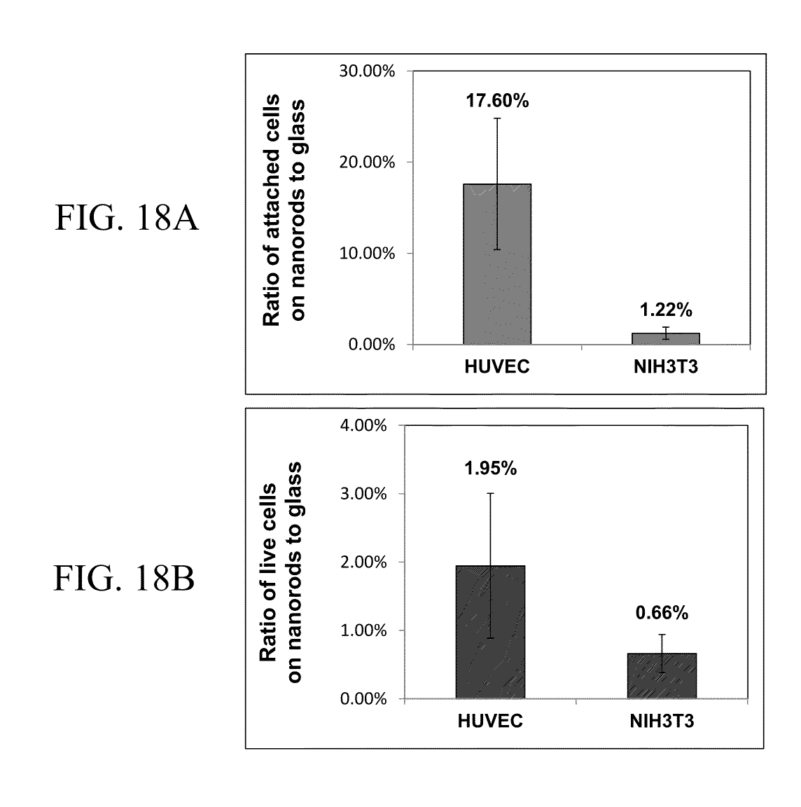

The number of cells adherent on SiO₂ coated nanorods was significantly reduced (a reduction of 98% in fibroblasts, 82% in HUVECs) compared to cells on glass (FIG. 18A) after 24 hour culture. Next, a live/dead viability/cytotoxicity kit for mammalian cells was used for quantifying adherent cell viability. The decrease in viability in cells on nanorods compared to that on glass was dramatic (FIG. 18B) with only one or two cells surviving on the SiO₂ nanorods for every 100 viable cells on glass. By culturing cells on glass in media that was incubated for 1 day, 3 days and 7 days with the nanorods, we confirmed that the cell death was not due to toxicity of unknown dissolving material from the nanorods (data not shown). These results suggest that densely packed nanorods have excellent anti-fouling potential by virtue of their topology.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

TABLE 1
Average area of cell spreading on ZnO flat substrate and ZnO nanorods
(Average area ± Standard Error of the Mean (μm²))

| | NIH 3T3 | HUVEC | BCE |
|---|---|---|---|
| ZnO flat substrate | (1.44E+03) ± (2.76E+02) | (1.73E+03) ± (2.35E+02) | (4.18E+03) ± (7.49+02) |
| ZnO nanorods | (4.73E+02) ± (7.44E+01) | (6.02E+02) ± (6.94E+01) | (1.22E+03) ± (1.17E+02) |

The differences of cell spreading area on ZnO flat substrate versus ZnO nanorods were statistically significant. (n=10, for NIH 3T3 and BCE, p<0.005 and for HUVEC p<0.0005).

### REFERENCES

Agren M S, Mirastschijski U. The release of zinc ions from and cytocompatibility of two zinc oxide dressings. J Wound Care 2004; 13(9):367-9.
Agren M S. Zinc in wound repair. Archives of Dermatology 1999; 135(10):1273-4.
Ainslie K M, Desai T A. Microfabricated implants for applications in therapeutic delivery, tissue engineering, and biosensing. Lab Chip. 2008; 8:1864-1878.
Ainslie K M, Sharma G, Dyer M A, Grimes C A, Pishko M V. Attenuation of protein adsorption on static and oscillating magnetostrictive nanowires. Nano Lett 2005; 5:1852-1856.
Al-Hilli S M, Willander M, Ost A, Stralfors P. ZnO nanorods as an intracellular sensor for pH measurements. J Appl Phys 2007; 102:084304.
Anderson J M, Rodriguez A, Chang D T. Foreign body reaction to biomaterials. Seminars in Immunology 2008; 20(2):86-100.
Anderson J M. Biological responses to materials Annual Review of Materials Research 2001; 31:81-110.
Anderson J M. Inflammation, Wound Healing and the Foreign Body Response. In: Buddy D. Ratner Allan S. Hoffman FJSJEL, editor. Biomaterials Science. Second Edition ed. Academic Press, 2004; p. 296-304.
Andersson A S, Backhed F, von Euler A, Richter-Dahlfors A, Sutherland D, Kasemo B. Nanoscale features influence epithelial cell morphology and cytokine production. Biomaterials 2003; 24:3427-3436.
Arnold M, Cavalcanti-Adam E A, Glass R, Blummel J, Eck W, Kantlehner M, Kessler H, Spatz J P. Activation of integrin function by nanopatterned adhesive interfaces. Chemphyschem 2004; 5:383-388.
Asuri P, Karajanagi S S, Kane R S, Dordick J S. Polymer-nanotube-enzyme composites as active antifouling films. Small 2007; 3:50-53.
Benzeev A, Farmer S R, Penman S. Protein-Synthesis Requires Cell-Surface Contact While Nuclear Events Respond to Cell-Shape in Anchorage-Dependent Fibroblasts. Cell 1980; 21:365-372.
Bershadsky A, Kozlov M, Geiger B. Adhesion-mediated mechanosensitivity: a time to experiment, and a time to theorize. Curr Opin Cell Biol 2006; 18:472-481.
Bodrumlu E. Biocompatibility of retrograde root filling materials: a review. Aust Endod J 2008; 34(1):30-5.
Brinkman S F, Johnston W D. Acute toxicity of aqueous copper, cadmium, and zinc to the mayfly Rhithrogena hageni. Arch Environ Contam Toxicol 2008; 54:466-472.
Cai X M, Cheung K Y, Djuri\check{s}ié A B, Xie, M H. Growth of cubic and hexagonal InN nanorods. Materials Letters 2007; 61:1563-1566.
Cavalcanti-Adam E A, Volberg T, Micoulet A, Kessler H, Geiger B, Spatz J P. Cell spreading and focal adhesion dynamics are regulated by spacing of integrin ligands. Biophys J 2007; 92:2964-2974.
Chen C S, Mrksich M, Huang S, Whitesides G M, Ingber D E. Geometric control of cell life and death. Science 1997; 276:1425-1428.
Choi C H, Hagvall S H, Wu B M, Dunn J C, Beygui R E, CJ C J K. Cell interaction with three-dimensional sharp-tip nanotopography. Biomaterials 2007; 28: 1672-1679.
Chu B H, Leu, L. C., Chang, C. Y., Lugo, F., Norton, D., Lele, T., Keselowsky, B., Pearton, S. J., Ren, F. Conformable coating of SiO₂ on hydrothermally grown ZnO nanorods. Appl Phys Lett 2008; 93:233111.
Colon G, Ward B C, Webster T J. Increased osteoblast and decreased Staphylococcus epidermidis functions on nanophase ZnO and TiO2. Journal of Biomedical Materials Research Part A 2006; 78A(3):595-604.
Colyar M. Unna boot application. Adv Nurse Pract 2006; 14(8).
Cunnick J, Kaur P, Cho Y, Groffen J, Heisterkamp N. Use of bone marrow-derived macrophages to model murine innate immune responses. Journal of Immunological Methods 2006 Apr. 20; 311(1-2):96-105.
Dalby M J, Gadegaard N, Wilkinson C D W. The response of fibroblasts to hexagonal nanotopography fabricated by electron beam lithography. Journal of Biomedical Materials Research Part A 2008; 84A(4):973-9.
Dalby M J, Riehle M O, Johnstone H, Affrossman S, Curtis A S. In vitro reaction of endothelial cells to polymer demixed nanotopography. Biomaterials 2002; 23:2945-2954.
Dalby M J, Riehle M O, Sutherland D S, Agheli H, Curtis A S. Morphological and microarray analysis of human fibroblasts cultured on nanocolumns produced by colloidal lithography. Eur Cell Mater 2005;9:1-8.
Dickinson R B, Caro L, Purich D L. Force generation by cytoskeletal filament end-tracking proteins. Biophys J 2004; 87:2838-2854.
Diehl K A, Foley J D, Nealey P F, Murphy C J. Nanoscale topography modulates corneal epithelial cell migration. J Biomed Mater Res A 2005;75:603-61 1.
Dillmore W S, Yousaf M N, Mrksich M. A photochemical method for patterning the immobilization of ligands and cells to self-assembled monolayers. Langmuir 2004; 20:7223-7231.
Dorfman A, Kumar N, Hahm J. Nanoscale ZnO-enhanced fluorescence detection of protein interactions. Adv Mater 2006; 18:2685-2690.
Dorfman A, Kumar N, Hahm J I. Highly sensitive biomolecular fluorescence detection using nanoscale ZnO platforms. Langmuir 2006; 22:4890-4895.
Elias K L, Price R L, Webster T J. Enhanced functions of osteoblasts on nanometer diameter carbon fibers. Biomaterials 2002; 23(15):3279-87.
Flemming R G, Murphy C J, Abrams G A, Goodman S L, Nealey P F. Effects of synthetic micro- and nano-structured surfaces on cell behavior. Biomaterials 1 999; 20:573-588.
Garibaldi S, Brunelli C, Bavastrello V, Ghigliotti G, Nicolini C. Carbon nanotube biocompatibi lity with cardiac muscle cells. Nanotechnol 2006; 17:391-397.
Genove E, Shen C, Zhang S G, Semino C E. The effect of functionalized self-assembling peptide scaffolds on human aortic endothelial cell function. Biomaterials 2005; 26:3341-3351.
Girard P P, Cavalcanti-Adam E A, Kemkemer R, Spatz J P. Cellular chemomechanics at interfaces: sensing, integration and response. Soft Matter 2007; 3:307-326.
Gojova A, Guo B, Kota R S, Rutledge J C, Kennedy I M, Barakat A I. Induction of inflammation in vascular endothelial cells by metal oxide nanoparticles: Effect of particle composition. Environmental Health Perspectives 2007; 115(3):403-9.
Goldberg M, Langer R, Jia X Q. Nanostructured materials for applications in drug delivery and tissue engineering. J Biomater Sci Polym Ed 2007; 18:241-268.
Gonsalves K E, Halberstadt C R, Laurencin C T, Nair L S. Biomedical Nanostructures. New York: John Wiley & Sons Inc; 2007.
Graeter S V, Huang J, Perschmann N, Lopez-Garcia M, Kessler H, Ding J, Spatz J P. Mimicking cellular environments by nanostructured soft interfaces. Nano Lett 2007; 7:141 3-1418.
Greene L E, Law M, Goldberger J, Kim F, Johnson J C, Zhang Y, Saykally R J, Yang P. Low-temperature wafer-scale production of ZnO nanowire arrays. Angew Chem Int Ed Engl 2003; 42:3031-3034.
Huang Z B, Zheng X, Yan D H, Yin G F, Liao X M, Kang Y Q, et al. Toxicological effect of ZnO nanoparticles based on bacteria. Langmuir 2008; 24(8):4140-4.
Ingber D E. Fibronectin Controls Capillary Endothelial-Cell Growth by Modulating Cell-Shape. Proc Natl Acad Sci USA 1990; 87:3579-3583.
Jeng H A, Swanson J. Toxicity of metal oxide nanoparticles in mammalian cells. Journal of Environmental Science and Health Part A-Toxic/Hazardous Substances & Environmental Engineering 2006; 41(12):2699-711.
Jia X, He W, Zhang X, Zhao H, Li Z, Feng Y. Microwave-assisted synthesis of anatase TiO₂ nanorods with mesopores. Nanotechnology 2007; 18:1-7.
Joo J, Kwon S G, Yu T, Cho M, Lee J, Yoon J, Hyeon T. Large-scale synthesis of TiO₂ nanorods via nonhydrolytic sol-gel ester elimination reaction and their application to photocatalytic inactivation of E. coli. J. Phys. Chem. B 2005; 109:15297-15302.
Kang B S, Pearton S J, Ren F. Low temperature (<100 degrees C.) patterned growth of ZnO nanorod arrays on Si. Appl Phys Lett 2007; 90:084104.
Kang B S, Ren F, Heo Y W, Tien L C, Norton D P, Pearton S J. pH measurements with single ZnO nanorods integrated with a microchannel. Appl Phys Lett 2005; 86:112105.
Kang B S, Wang H T, Ren F, Pearton S J, Morey T E, Dennis D M, et al. Enzymatic glucose detection using ZnO nanorods on the gate region of AlGaN/GaN high electron mobility transistors. Applied Physics Letters 2007; 91(25).
Kao W J, Hubbell J A, Anderson J M. Protein-mediated macrophage adhesion and activation on biomaterials: a model for modulating cell behavior. Journal of Materials Science-Materials in Medicine 1999; 10(10-11):601-5.
Karp J M, Yeh J, Eng G, Fukuda J, Blumling J, Suh K Y, et al. Controlling size, shape and homogeneity of embryoid bodies using poly(ethylene glycol) microwells. Lab Chip 2007; 7:786-794.
Karuri N W, Liliensiek S, Teixeira A I, Abrams G, Campbell S, Nealey P F, Murphy C J. Biological length scale topography enhances cell-substratum adhesion of human corneal epithelial cells. J Cell Sci 2004; 117:3153-3164.
Keselowsky B G, Bridges A W, Burns K L, Tate C C, Babensee J E, LaPlaca M C, et al. Role of plasma fibronectin in the foreign body response to biomaterials. Biomaterials 2007; 28(25):3626-31.
Keselowsky B G, Collard D M, Garcia A J. Surface chemistry modulates fibronectin conformation and directs integrin binding and specificity to control cell adhesion. Journal of Biomedical Materials Research Part A 2003; 66A(2):247-59.
Khademhosseini A, Langer R, Borenstein J, Vacanti J P. Microscale technologies for tissue engineering and biology. Proc Natl Acad Sci USA 2006;103:2480-2487.
Khang D, Lu J, Yao C, Haberstroh K M, Webster T J. The role of nanometer and sub-micron surface features on vascular and bone cell adhesion on titanium. Biomaterials 2008; 29:970-983.
Kietzmann M. Improvement and retardation of Round healing: effects of pharmacological agents in laboratory animal studies. Veterinary Dermatology 1999; 10(2):83-8.
Kim W, Ng J K, Kunitake M E, Conklin B R, Yang P. Interfacing silicon nanowires with mammalian cells. J Am Chem Soc 2007a; 129:7228-7229.
Kim, J Y, Khang D, Lee J E, Webster T J. Decreased Macrophage Density on Carbon Nanofiber Patterns. Mater Res Soc Symp Proc 2007b; 950.
Koc Y, de Mello A J, McHale G, Newton M I, Roach P, Shirtcliffe N J. Nano-scale superhydrophobicity: suppression of protein adsorption and promotion of flow-induced detachment. Lab Chip 2008; 8:582-586.
Kryliouk O, Park H J, Won Y S, Anderson T, Davydov A, Levin I, Kim J H, Freitas J A. Controlled synthesis of single-crystalline InN nanorods. Nanotechnology 2007; 18:1-6.
Kuo S Y, Kei C C, Chao C K, Hsiao C N, Lai F-I, Kuo H C, Hsieh W F, Wang S C. Catalyst-free GaN nanorods grown by metalorganic molecular beam epitaxy. Proceedings of 2005 5^{th} IEEE Conference on Nanotechnology, Nagoya, Japan, July 2005
Lauffenburger D A, Horwitz A F. Cell migration: A physically integrated molecular process. Cell 1996; 84:359-369.
Lee J Y, Kang B S, Hicks B, Chancellor T F, Chu B H, Wang H T, et al. The control of cell adhesion and viability by zinc oxide nanorods. Biomaterials 2008; 29(27):3743-3749.
Lele T P, Pendse J, Kumar S, Salanga M, Karavitis J, Ingber D E. Mechanical forces alter zyxin unbinding kinetics within focal adhesions of living cells. J Cell Physiol 2006; 207: 187-194.
Lenhert S, Meier M B, Meyer U, Chi L F, Wiesmann H P. Osteoblast alignment, elongation and migration on grooved polystyrene surfaces patterned by LangmuirBlodgett lithography. Biomaterials 2005; 26:563-570.
Liliensiek S J, Campbell S, Nealey P F, Murphy C J. The scale of substratum topographic features modulates proliferation of corneal epithelial cells and corneal fibroblasts. J Biomed Mater Res A 2006; 79A:185-192.
Liliental-Weber Z, Park H J, Mangum J, Anderson T, Kryliouk O. InN nanrods grown on different planes of Al₂O₃. Microsc. Microanal. 2007; 13(Suppl 2):1-2.
Lin W S, Xu Y, Huang C C, Ma Y F, Shannon K B, Chen D R, et al. Toxicity of nano- and micro-sized ZnO particles in human lung epithelial cells. J Nanopart Res 2009; 11:25-39.
Ma Z W, He W, Yong T, Ramakrishna S. Grafting of gelatin on electrospun poly(caprolactone) nanofibers to improve endothelial cell spreading and proliferation and to control cell orientation. Tissue Eng 2005; 11:1149-1158.
Meng J, Meng J, Duan J, Kong H, Li L, Wang C, et al. Carbon nanotubes conjugated to tumor lysate protein enhance the efficacy of an antitumor immunotherapy. Small 2008; 4:1364-1370.
Michikawa M, Yanagisawa K. Apolipoprotein E4 induces neuronal cell death under conditions of suppressed de novo cholesterol synthesis. J Neurosci Res 1 998; 54:58-67.
Mooney D, Hansen L, Vacanti J, Langer R, Farmer S, Ingber D. Switching from Differentiation to Growth in Hepatocytes—Control by Extracellular-Matrix. J Cell Physiol 1992; 151:497-505.
Moriarty P. Nanostructured materials. Reports on Progress in Physics 2001; 64(3):297-381.
Nel A, Xia T, Madler L, Li N. Toxic potential of materials at the nanolevel. Science 2006; 311(5761):622-7.
Nohynek G J, Lademann J, Ribaud C, Roberts M S. Grey goo on the skin? Nanotechnology, cosmetic and sunscreen safety. Critical Reviews in Toxicology 2007; 37(3):251-77.
Pacholski C, Kornowski A, Weller H. Self-assembly of ZnO: From nanodots, to nanorods. Angew Chem Int Ed 2002; 41:1188-1 991.
Papageorgiou, Chu. Chloroxylenol and zinc oxide containing cream (Nels cream®) vs. 5% benzoyl peroxide cream in the treatment of acne vulgaris. A double-blind, randomized, controlled trial. Clinical and Experimental Dermatology 2001; 25(1):16-20.
Park H, Cannizzaro C, Vunjak-Novakovic G, Langer R, Vacanti C A, Farokhzad O C. Nanofabrication and microfabrication of functional materials for tissue engineering. Tissue Eng 2007a; 13:1867-1877.
Park J, Bauer S, von der Mark K, Schmuki P. Nanosize and vitality: TiO₂ nanotube diameter directs cell fate. Nano Lett 2007b; 7:1686-1691.
Parker K K, Brock A L, Brangwynne C, Mannix R J, Wang N, Ostuni E, Geisse N A, Adams J C, Whitesides G M, Ingber D E. Directional control of lamellipodia extension by constraining cell shape and orienting cell tractional forces. FASEB J 2002; 16:1195-1204.
Patzig C, Rauschenbach B, Fuhrmann B, Leipner H S. Growth of Si nanorods in honeycomb and hexagonal-closed-packed arrays using glancing angle deposition. Journal of Applied Physics 2008; 103:1-6.
Pearton S J, Lele T, Tseng Y, Ren F. Penetrating living cells using semiconductor nanowires. Trends Biotechnol 2007; 25:48 1-482.
Petty R T, Li H W, Maduram J H, Ismagilov R, Mrksich M. Attachment of cells to islands presenting gradients of adhesion ligands. J Am Chem Soc 2007; 129:8966-8967.
Price R L, Waid M C, Haberstroh K M, Webster T J. Selective bone cell adhesion on formulations containing carbon nanofibers. Biomaterials 2003; 24(11):1877-87.
Puckett S, Pareta R, Webster T J. Nano rough micron patterned titanium for directing osteoblast morphology and adhesion. Int J Nanomedicine 2008; 3:229-241.
Ratner B D, Hoffman A S, Schoen F J, Lemmons J E. Biomaterials science: an introduction to materials in medicine. London, UK: Elsevier Academic Press; 2004.
Ratner B D. Reducing capsular thickness and enhancing angiogenesis around implant drug release systems. Journal of Controlled Release 2002; 78(1-3):211-8.
Re F, Zanetti A, Sironi M, Polentarutti N, Lanfrancone L, Dejana E, Colotta F. Inhibition of anchorage-dependent cell spreading triggers apoptosis in cultured human endothelial cells. J Cell Biol 1994; 127:537-546.
Reddy K. M BJWDGHCPA. Selective toxicity of zinc oxide nanoparticles to prokaryotic and eukaryotic systems. Applied Physics Letters 90, 213902-1-213902-3. 2007.
Sato M, Aslani A, Sambito M A, Kalkhoran N M, Slamovich E B, Webster T J. Nanocrystalline hydroxyapatite/titania coatings on titanium improves osteoblast adhesion. J Biomed Mater Res A 2008; 84:265-272.
Sofia S J, Premnath V V, Merrill E W. Poly(ethylene oxide) Grafted to Silicon Surfaces: Grafting Density and Protein Adsorption. Macromolecules 1998; 31:5059-5070.
Spatz J P, Geiger B. Molecular engineering of cellular environments: cell adhesion to nano-digital surfaces. Methods Cell Biol 2007; 83:89-1 11.
Stanley E. R. Murine Bone Marrow-derived Macrophages. Basic Cell Culture Protocols. Second Edition ed. Humana press; 1997. p. 301-4.
Sun T, Tan H, Han D, Fu Q, Jiang L. No platelet can adhere—largely improved blood compatibility on nanostructured superhydrophobic surfaces. Small 2005; 1:959-963.
Sun X W, Kwok H S. Optical properties of epitaxially grown zinc oxide films on sapphire by pulsed laser deposition. Journal of Applied Physics 1999; 86(1):408-11.
Tanemura M, Kobayashi M, Kudo M, Yamauchi H, Okita T, Miao L, Tanemura S. Fabrication of densely distributed Si nanorods by Ar⁺-ion bombardment. Surface Science 2006; 600:3668-3672.
Tarnow P, Agren M, Steenfos H, Jansson J O. Topical Zinc-Oxide Treatment Increases Endogenous Gene-Expression of Insulin-Like Growth-Factor-I in Granulation-Tissue from Porcine Wounds. Scandinavian Journal of Plastic and Reconstructive Surgery and Hand Surgery 1994; 28(4):255-9.
Teixeira A I, Abrams G A, Bertics P J, Murphy C J, Nealey P F. Epithelial contact guidance on well-defined micro- and nanostructured substrates. J Cell Sci 2003; 1 16:1881-1 892.
Teixeira A I, McKie G A, Foley J D, Bertics P J, Nealey P F, Murphy C J. The effect of environmental factors on the response of human corneal epithelial cells to nanoscale substrate topography. Biomaterials 2006; 27:3945-3954.
Thakar R G, Chown M G, Patel A, Peng L, Kumar S, Desai T A. Contractility-dependent modulation of cell proliferation and adhesion by microscale topographical cues. Small. 2008; 4:1416-1424.
Tran P, Webster T J. Enhanced osteoblast adhesion on nanostructured selenium compacts for anti-cancer orthopedic applications. Int J Nanomedicine 2008; 3:391-396.
Venugopal J, Low S, Choon A T, Ramakrishna S. Interaction of cells and nanofiber scaffolds in tissue engineering. J Biomed Mater Res B Appl Biomater 2008; 84:34-48.
Washburn N R, Yamada K M, Simon C G, Jr., Kennedy S B, Amis E J. High-throughput investigation of osteoblast response to polymer crystallinity: influence of nanometerscale roughness on proliferation. Biomaterials 2004; 25:1215-1224.
Wiggins M J, Wilkoff B, Anderson J M, Hiltner A. Biodegradation of polyether polyurethane inner insulation in bipolar pacemaker leads. Journal of Biomedical Materials Research 2001; 58(3):302-7.
WojciakStothard B, Curtis A, Monaghan W, Macdonald K, Wilkinson C. Guidance and activation of murine macrophages by nanometric scale topography. Experimental Cell Research 1996; 223(2):426-35.
Wooley P H, Schwarz E M. Aseptic loosening. Gene Therapy 2004; 11(4):402-7.
Yan P, Qin D, An Y K, Li G Z, Xing J, Liu J J. In situ synthesis and characterization of GaN nanorods through thermal decomposition of pre-grown GaN films. Nanotechnology 2008; 19:1-5.
Yeo W S, Mrksich M. Electroactive self-assembled monolayers that permit orthogonal control over the adhesion of cells to patterned substrates. Langmuir 2006; 22:10816-10820.
Yim E K F, Reano R M, Pang S W, Yee A F, Chen C S, Leong K W. Nanopatterninduced changes in morphology and motility of smooth muscle cells. Biomaterials 2005; 26:5405-541 3.
Zhu X Y, Mills K L, Peters P R, Bahng J H, Liu E H, Shim J, Naruse K, Csete M E, Thouless M D, Takayama S. Fabrication of reconfigurable protein matrices by cracking. Nature Materials 2005; 4:403-406.

1-9. (canceled)

10. A method for preventing, inhibiting, and/or reducing adhesion, growth, and/or survival of a cell to a substrate surface of a device, product, apparatus, structure, or material, comprising providing a nanorod composed of ZnO, TiO₂, Si, InN, or GaN, or a substrate comprising the nanorod, to the device, product, apparatus, structure, or material.

11. The method of claim 10, wherein the cell is a bacterial cell.

12. The method of claim 10, wherein the cell is a mammalian cell.

13. The method of claim 12, wherein the mammalian cell is a tumor or cancer cell.

14. The method of claim 13, wherein the tumor or cancer cell is from a tumor or cancer of the bone, breast, kidney, mouth, larynx, esophagus, stomach, testis, cervix, head, neck, colon, ovary, lung, bladder, skin, liver, muscle, pancreas, prostate, blood cells, or brain.

15. The method of claim 12, wherein the mammalian cell is a platelet, fibroblast, endothelial cell, osteoclast, osteoblast, or macrophage.

16-20. (canceled)

21. The method of claim 10, wherein the nanorod is coated with SiO₂ and/or TiO₂.

22. The method of claim 10, wherein the nanorod is from about 100 nm to about 5 μm in height; and/or is from about 5 nm to about 150 nm in diameter; and/or is spaced apart from between about 5 nm and about 150 nm.

23. The method of claim 10, wherein the surface of the nanorod is functionalized with a moiety to bind to or act as an attractant for a target cell, compound, or molecule.

24. The method of claim 23, wherein the moiety is an antibody, receptor, peptide, nucleic acid, or aptamer that has binding specificity for the target cell, compound, or molecule.

25. The method of claim 10, wherein the nanorod is coated or conjugated with one or more cellular toxins, or with a compound or drug, or with a radioactive element or molecule.

26. The method of claim 25, wherein the cellular toxin is released in an intracellular environment.

27. The method of claim 25, wherein the cellular toxin is ricin, mitomycin C, cisplatin, botulinum toxin, anthrax toxin, or aflatoxin.

28. The method of claim 25, wherein the cellular toxin is attached to a cleavable moiety that can be cleaved by a molecule or molecules present inside a cell.

29. The method of claim 25, wherein the compound or drug is a protein, peptide, or nucleic acid.

30. The method of claim 25, wherein the compound or drug is heparin.

31. The method of claim 14, wherein the blood cells are lymphocytes.

32. The method of claim 10, wherein the nanorods are in vertically aligned monolayers on the substrate surface.

33. The method of claim 10, wherein the substrate is a glass, plastic, fiberglass, wood, rubber, metal, alloy, ceramic, cloth, polymer, concrete, silicon, or paint.

34. The method of claim 10, wherein the nanorod is composed of ZnO coated with SiO₂, TiO₂, or SiO₂ and TiO₂.
